(19) United States
(12) Patent Application Publication
(10) Pub. No.: US 2023/0084354 A1
(43) Pub. Date: Mar. 16, 2023

(54) NUTRACEUTICAL EXTRACTS OF HIPPOPHAE RHAMNOIDES AND TERMINALIA CHEBULA AND USES THEREOF

(71) Applicant: Biomics, LLC, Austin, TX (US)

(72) Inventors: Christian DRAPEAU, Austin, TX (US); Gitte S. JENSEN, Austin, TX (US)

(73) Assignee: Biomics, LLC, Austin, TX (US)

(21) Appl. No.: 17/759,542

(22) PCT Filed: Sep. 18, 2020
(86) PCT No.: PCT/US2020/051624

§ 371 Date: Jul. 27, 2022

Related U.S. Application Data
(60) Provisional application No. 62/966,417, filed on Jan. 27, 2020.

(51) Int. Cl. A61K 36/73; A61K 35/748; A61K 36/886; A61K 36/03; A61K 36/258; A61K 31/716; A61K 9/00; A61K 9/16; A61P 25/00

(52) U.S. Cl. CPC A61K 36/73; A61K 35/748; A61K 36/886; A61K 36/03; A61K 36/258; A61K 31/716; A61K 9/0053; A61K 9/16; A61P 25/00

(57) ABSTRACT

The present disclosure is directed to compositions and methods for the administration of a nutraceutical. The present disclosure is also directed to compositions and methods for the trafficking of stem cells.

This application claims the benefit of U.S. Provisional Patent Application No. 62/966,417, filed Jan. 27, 2020, the entirety of which is incorporated herein by reference.

### BACKGROUND OF THE INVENTION

### 1. Field of the Invention

The present application relates generally to the field of nutraceuticals. More particularly, it concerns compositions that may be used for mobilization of stem cells.

### 2. Description of Related Art

Stem cells (SC) are defined as cells with the capacity to self-replicate throughout the entire life of an organism and to differentiate into various cell types of the body. Two well-known types of stem cells are embryonic stem cells and adult stem cells. Embryonic stem cells (ESCs) are extracted from 5-10 day old embryos called blastulas. Once isolated, ESCs can be grown in vitro and led to differentiate into various types of tissue cell (such as heart cells, liver cells, nervous cells, and kidney cells), after which they can be injected in specific tissues in order to regenerate the tissue.

Adult stem cells (ASCs) are undifferentiated or primitive cells that can self-renew and differentiate into specialized cells of various tissues and are found in any living organism afterbirth. ASCs have been isolated from various tissues such as the liver (oval cells), the intestine (intestinal crypt stem cells), muscles (satellite cells), the brain (neural stem cells), and fat tissue (adipocyte stem cells). Umbilical cord stem cells and placental stem cells are considered ASCs.

The role of ASCs found in tissues (tissue stem cells) is to maintain and repair the tissue in which they are found, although recent studies have reported that ASCs from one tissue may have the ability to develop into cell types characteristic of other tissues. For example, oval cells in the liver were shown in vitro to have the ability to become insulin-producing pancreatic cells. Nevertheless, the general view is that local stem cells are primarily involved in minor repair of the tissue in which they reside. In the case of significant injury or degeneration, the number of new tissue cells found in healing tissue far exceeds the capacity of local stem cells to duplicate and differentiate, suggesting that stem cells coming from other sites must be involved in the process of repair.

Although many tissues contain their own specific population of tissue stem cells, certain ASCs of key interest are those primarily found in the bone marrow and blood. Tissue stem cells are traditionally believed to be limited in their ability to differentiate into other tissues. However bone marrow stem cells (BMSC) have been shown to have the capacity to become cells of other tissues.

Different stem cells in the body, whether BMSCs, HSCs, marrow stromal cells (MSCs), multipotent adult progenitor cells (MAPCs), very small embryonic-like stem cells (VSEL), epiblast-like stem cell (ELSC) or blastomere-like stem cell (BLSC), constitute a broad component of the body's natural healing system. Since stem cells are capable of differentiating into a broad variety of cell types, they play an important role in the healing and regenerative processes of various tissues and organs. Bone marrow stem cells, including marrow stromal cells (MSCs), are released from tissues of origin, and circulate in a subject's circulatory system to migrate into various organs and tissues to become mature, terminally differentiated cells. Therefore, enhancement of stem cell trafficking (i.e., release, circulation, homing and/or migration) may amplify these physiological processes and provide potential therapies for various pathologies. Existing methods of promoting stem cell mobilization suffer from significant drawbacks, including poor kinetic performance, high cost, inconvenient methods of administration and undesirable side effects. One leading approach, injection of granulocyte colony-stimulating factor (G-CSF) or recombinant forms thereof, requires days to achieve peak circulating HSC numbers. The opposite problem exists with administration of interleukin-8 (IL-8), which acts only within minutes and has a short-lived effect on elevating circulating HSC levels in the bloodstream. G-CSF and a different molecule, CXCR4 antagonist AMD3100, can have significant side effects, including hemorrhaging, stroke, rupturing of the spleen, bloody sputum, bone disorders, among others. Thus, there is a need in the art for an effective and convenient method for delivering stem cell mobilization agents to human subjects, to obtain positive clinical benefits without side effects and at a reduced cost.

Accordingly, in some aspects, the compositions and methods disclosed herein may enhance the release and homing and/or migration of stem cells within the body to promote healing and treatment of damaged tissues, as well as aid in the regeneration of tissues that suffer from some level of cellular loss, which may result in greater vitality and reduced incidence of disease.

### SUMMARY OF THE INVENTION

In some aspects, the present disclosure provides pharmaceutical compositions comprising: one or more of the following components selected from the group consisting of: a Hippophae rhamnoides extract, a Ribes spp. Extract; a Aphanrizomenon flos aquae (AFA) extract; a Aloe extract; a Fucus vesiculosus extract; a Panax notoginseng extract; a beta-glucan and a pharmaceutically acceptable carrier. In some embodiments, the composition comprises a Hippophae rhamnoides extract and at least one additional component selected from: a Ribes spp. extract; a Aphanizomenon flos aquae (AFA) extract; a Aloe extract; a Fucus vesiculosus extract; a Panax notoginseng extract; and a beta-glucan. In some embodiments, the composition comprises a Hippophae rhamnoides extract and at least two additional components selected from: a Ribes spp. extract; an Aphanizomenon flos aquae (AFA) extract; a Aloe extract; a Fucus vesiculosus extract; a Panax notoginseng extract; and a beta-glucan. In some embodiments, the composition comprises a Hippophae rhamnoides extract and at least three, four or five additional components selected from: a Ribes spp. extract; an Aphanizomenon flos aquae (AFA) extract; a Aloe extract; a Fucus vesiculosus extract; a Panax notoginseng extract; and a beta-glucan. In some embodiments, the composition comprises a Hippophae rhamnoides extract and at least one, two, three, four or five additional components selected from: an Aphanizomenon flos aquae (AFA) extract; an Aloe extract; a Fucus vesiculosus extract; a Panax notoginseng extract; and a beta-glucan.

In some embodiments, the pharmaceutical composition comprises a Hippophae rhamnoides fruit or a Hippophae rhamnoides leaf extract. In some embodiments, the pharmaceutical composition comprises a Hippophae rhamnoides leaf extract. In some embodiments, the pharmaceutical composition comprises a Hippophae rhamnoides fruit extract. In some embodiments, the Hippophae rhamnoides fruit extract or the Hippophae rhamnoides leaf extract is an extract enriched for polyphenol and/or proanthocyanidin compounds. In some embodiments, the composition is a single unit dosage and comprises 150-1000 mg of Hippophae rhamnoides extract.

In some embodiments, the composition comprises an Aphanizomenon flos aquae (AFA) extract. In some embodiments, the composition is a single unit dosage and comprises 250-1000 mg of Aphanizomenon flos aquae (AFA) extract.

In some embodiments, the composition comprises a Fucus vesiculosus extract. In some embodiments, the composition is a single unit dosage and comprises 150-1000 mg of a Fucus vesiculosus extract. In some embodiments, the Fucus vesiculosus extract comprises at least 20%, 25%, 30%, 35%, or 40% phlorotannins.

In some embodiments, the composition comprises a Panax notoginseng extract. In some embodiments, the composition is a single unit dosage and comprises 100-500 mg of a Panar notoginseng extract. In some embodiments, the Panax notoginseng extract comprises at least 20%, 25%, 30%, 35%, or 40% saponins.

In some embodiments, the compositions comprise a beta-glucan. In some embodiments, the composition is a single unit dosage and comprises 50-500 mg of a beta-glucan.

In some embodiments, the compositions comprise colostrum. In some embodiments, the composition is a single unit dosage and comprises 5-100 mg of colostrum.

In some embodiments, the compositions comprise a Hippophae rhamnoides extract; an Aphanizomenon flos aquae (AFA) extract; an Aloe extract; a Fucus vesiculosus extract; a Panax notoginseng extract; and a beta-glucan. In some embodiments, the compositions comprise a Hippophae rhamnoides extract; a Ribes spp. extract; an Aphanizomenon flos aquae (AFA) extract; an Aloe extract; a Fucus vesiculosus extract; a Panar notoginseng extract; and a beta-glucan.

In some embodiments, the compositions comprise a Ribes spp. extract.

In some embodiments, the compositions comprise a Ribes nigrum and/or Ribes rubrum fruit extract. In some embodiments, the Ribes nigrum and/or Ribes rubrum fruit extract is enriched for polyphenol compounds. In some embodiments, the pharmaceutical composition comprises a polyphenol extract of Ribes nigrum and or Ribes rubrum. In some embodiments, the composition further comprises an Aloe extract, an Fucus vesiculosus extract, and/or an Aphanizomenon fjlos aquae extract. In some embodiments, the composition comprises an Fucus vesiculosus extract comprising fucoidan and at least 5%, 10%, 20% or 30% phlorotannins content. In some embodiments, the composition comprises an Aloe extract that has been enriched for poly acetyl mannan content. In some embodiments, the Aloe extract comprises at least 10%, 15%, or 20% poly acetyl mannans. In some embodiments, the composition is a single unit dosage and comprises 100-400 mg of Aloe extract.

In some embodiments, the compositions comprise a Terminalia chebula extract. In some embodiments, the compositions comprise a Terminalia chebula fruit extract or a Terminalia chebula leaf extract. In some embodiments, the compositions comprise a Terminalia chebula leaf extract. In some embodiments, the compositions comprise a Terminalia chebula fruit extract. In some embodiments, the Terminalia chebula fruit or Terminalia chebula leaf extract is enriched for tannin compounds. In some embodiments, the pharmaceutical composition comprises a tannin extract of Terminalia chebula. In some embodiments, the Terminalia chebula fruit extract or the Terminalia chebula leaf extract comprises at least 10%, 20%, 30% or 40% tannins.

In some aspects, the present disclosure provides pharmaceutical compositions comprising: one or more of the following components selected from the group consisting of: a Hippophae rhamnoides extract, Terminalia chebula extract, Panax notoginseng extract, and Ribes rubrum and nigrum extract; and a pharmaceutically acceptable carrier. In some embodiments, the pharmaceutical compositions are comprised of a Hippophae rhamnoides extract and a Terminalia chebula extract. In some embodiments, the pharmaceutical compositions are comprised of a Hippophae rhamnoides extract and a Panax notoginseng extract. In some embodiments, the pharmaceutical compositions are comprised of a Ribes rubrum and nigrum extract and a Panax notoginseng extract. In other embodiments, the pharmaceutical compositions comprise a Hippophae rhamnoides fruit extract. In further embodiments, the Hippophae rhamnoides fruit extract is an extract enriched for polyphenol and/or proanthocyanidin compounds. In some embodiments, the Hippophae rhamnoides fruit extract is enriched to from about 5%, 10%, 15%, 20%, 25%, 30%, 35%, to about 40% proanthocyanidin compounds. In some embodiments, the Hippophae rhamnoides fruit extract is enriched to about 30% of proanthocyanidin compounds.

In some embodiments, the pharmaceutical compositions comprise a Terminalia chebula fruit extract. In further embodiments, the Terminalia chebula extract is enriched for tannin compounds. In other embodiments, the pharmaceutical compositions comprise a tannin extract of Terminalia chebula. In some embodiments, the Terminalia chebula fruit extract comprises from about 5%, 10%, 15%, 20%, 25%, 30%, 35%, 40%, 45%, 50%, 55%, 60%, to about 65% tannins or any range derivable therein. In preferred embodiments, the Terminalia chebula fruit extract comprises at least 10%, 20%, 30% or 40% tannins. In some further aspects, a composition of the embodiments comprises a Terminalia chebula fruit extract and comprises a total tannin content of 1%-5%, 2%-10%, 3%-15% or 5%-20%.

In other embodiments, the pharmaceutical compositions comprise a Panax notoginseng root extract. In further embodiments, the Panar notoginseng root extract is an extract enriched for ginsenosides and saponins compounds. In some embodiments, the Panar notoginseng root extract is enriched to from about 5%, 10%, 15%, 20%, 25%, 30%, 35%, to about 40% of ginsenosides and saponins compounds. In some embodiments, the Panax notoginseng root extract is enriched to about 25% of ginsenosides and saponins compounds.

In other embodiments, the pharmaceutical compositions comprise a Ribes rubrum and nigrum fruit extract. In further embodiments, the Ribes rubrum and nigrum fruit extract is an extract enriched for polyphenol compounds. In some embodiments, the Ribes rubrum and nigrum fruit extract is enriched to from about 5%, 10%, 15%, 20%, 25%, 30%, 35%, to about 40% of polyphenol compounds. In some embodiments, the Ribes rubrum and nigrum fruit extract is enriched to about 20% of polyphenol compounds.

In some embodiments, the pharmaceutical compositions further comprise an Aloe extract (e.g., a high poly acetyl mannans extract), a Fucus vesiculosus extract, an Aphanizomenon flos aquae (AFA) extract (see, e.g., U.S. Pat. No. 7,651,690, which is incorporated herein by reference), and β-glucan extracted from Avena sativa, Euglena gracilis, or Agrobacterium. In some embodiments, the pharmaceutical compositions comprise an Fucus vesiculosus extract comprising phlorotannins and other polyphenols from about 1%, 5%, 10%, 20% to about 30% or any range derivable therein. In preferred embodiments the pharmaceutical compositions comprise an Fucus vesiculosus extract comprising phlorotannins and other polyphenols at least 5%, 10% or 20% content. In some embodiments, the pharmaceutical compositions comprise an Aloe extract that has been enriched for poly acetyl mannan content. In preferred embodiments, the Aloe extract comprises at least 10%, 15%, or 20% poly acetyl mannans. Thus, in some compositions of the embodiments, the poly acetyl mannan content is at least 0.5%. In preferred embodiments the β-glucan from Avena saliva or Euglena gracilis or Agrobacterium comprises from about 10%, 20%, 50% to about 85% or 95% of content. In preferred embodiments the β-glucan is from Euglena gracilis or Agrobacterium and comprises about 85% to 95% of content.

In some embodiments, the pharmaceutical compositions are sterile. In other embodiments, the pharmaceutical compositions are formulated for oral administration. In some embodiments, the pharmaceutical compositions are comprised in a capsule. In other embodiments, the capsule comprises an enteric coating. In some embodiments, the capsule comprises from about 25 to 50, 75, 100, 125, 150, 175, 200, 225, 250, 300, 350, 400, 450, 500, or 1000 mg of the composition or any range derivable therein. In preferred embodiments, the capsule comprises about 50 to 100, 200, 250 or 500 mg of the composition.

In some embodiments, the pharmaceutical compositions comprise between 1 and 2, 3, 4, 5, 10, 25, 50, 100, 250, 500, or 1000 mg of Hippophae rhamnoides extract or any range derivable therein. In preferred embodiments, the pharmaceutical compositions comprise between about 50 and 750 mg of Hippophae rhamnoides extract. In other embodiments, the pharmaceutical compositions comprise between about 1 and 2, 3, 4, 5, 10, 25, 50, 100, 250, 500, or 1000 mg of Terminalia chebula extract or any range derivable therein. In preferred embodiments, the pharmaceutical compositions comprise between about 50 and 750 mg of Terminalia chebula extract.

In some embodiments, the pharmaceutical compositions comprise between 1 and 2, 3, 4, 5, 10, 25, 50, 100, 250, 500, or 1000 mg of Panax notoginseng root extract or any range derivable therein. In preferred embodiments, the pharmaceutical compositions comprise between about 50 and 250 mg of Panax notoginseng root extract. In other embodiments, the pharmaceutical compositions comprise between about 1 and 2, 3, 4, 5, 10, 25, 50, 100, 250, 500, or 1000 mg of Ribes rubrum and nigrum fruit extract or any range derivable therein. In preferred embodiments, the pharmaceutical compositions comprise between about 50 and 750 mg of Ribes rubrum and nigrum fruit extract. In other embodiments, the pharmaceutical compositions comprise between about 1 and 2, 3, 4, 5, 10, 25, 50, 100, 250, 500, or 1000 mg of Fucus vesiculosus extract or any range derivable therein. In preferred embodiments, the pharmaceutical compositions comprise between about 50 and 250 mg of Fucus vesiculosus extract.

In some aspects, the present disclosure provides methods of increasing stem cell mobilization or homing in a subject, comprising administering to the subject a sufficient amount of a composition comprising a Hippophae rhamnoides extract and/or a Panax notoginseng extract and/or a Ribes rubrum and nigrum fruit extract and/or a Fucus vesiculosus extract to provide stem cell mobilization to the blood and/or homing from the blood to the tissues. In some embodiments, the compositions comprise a Hippophae rhamnoides fruit extract. In other embodiments, the mobilization and/or homing agent comprises a Hippophae rhamnoides leaf extract. In other embodiments, the mobilization and/or homing agent comprises a Hippophae rhamnoides fruit and/or leaf extract that is enriched for polyphenols and/or proanthocyanidins. In yet other embodiments, the mobilization and/or homing agent comprises a Panax notoginseng root extract. In some embodiments, the mobilization and/or homing agent comprises a Panax notoginseng root extract comprising about 5%, 10%, 15%, 20%, 25%, 30%, 35%, 40%, 45%, 50%, to about 55% saponins or any range derivable therein. In preferred embodiments, the mobilization and/or homing agent comprises a Panar notoginseng root extract comprising at least 10%, 20% or 50% tannins. In yet other embodiments, the mobilization and/or homing agent comprises a Ribes rubrum and nigrum fruit extract. In some embodiments, the mobilization and/or homing agent comprises a Ribes rubrum and nigrum fruit extract comprising about 5%, 10%, 15%, 20%, 25%, 30%, 35%, 40%, 45%, 50%, to about 55% polyphenols or any range derivable therein. In preferred embodiments, the mobilization and/or homing agent comprises a Ribes rubrum and nigrum fruit extract comprising at least 10%, 20% or 50% polyphenols.

In some embodiments, the compositions further comprise an Aloe extract, an Fucus vesiculosus extract, an AFA extract, and a β-glucan extract. In some embodiments, the pharmaceutical compositions comprise an Fucus vesiculosus extract comprising from about 1%, 10%, 20%, 50% to about 75% phlorotannins content or any range derivable therein. In preferred embodiments, the mobilization agent comprises a Fucus vesiculosus extract comprising at least 10%, 20% or 25% phlorotannins content.

In some embodiments, the stem cell comprises a bone marrow-derived stem cell (BMSC). In other embodiments, the stem cell comprises a hematopoietic stem cell (HSC). In some embodiments, the composition comprises oral administration. In other embodiments, oral administration is more than once a day. In yet other embodiments, oral administration is daily.

In some embodiments, the composition is comprised in a capsule or tablet. In other embodiments, the capsule or tablet comprises an enteric coating. In some embodiments, the capsule or tablet comprises from about 25 to 50, 75, 100, 125, 150, 175, 200, 225, 250, 300, 350, 400, 450, 500, 1000, or 2000 mg of the composition or any range derivable therein. In preferred embodiments, the capsule or tablet comprises about 50 to 100, 200, 250 or 500 mg of the composition. In still further preferred embodiments, the capsule or tablet comprises about 250 to 500, 750, or 1000 mg of the composition.

In some embodiments, the methods comprise administering between 1 and 2, 3, 4, 5, 10, 25, 50, 100, 250, 500, or 1000 mg of Hippophae rhamnoides extract or any range derivable therein. In preferred embodiments, the methods comprise administering about 50 to 750 mg of Hippophae rhamnoides extract. In some embodiments, the methods comprise administering between 1 and 2, 3, 4, 5, 10, 25, 50, 100, 250, 500, or 1000 mg of Panar notoginseng extract or any range derivable therein. In preferred embodiments, the methods comprise administering about 50 to 250 mg of Panax notoginseng extract. In some embodiments, the methods comprise administering between 1 and 2, 3, 4, 5, 10, 25, 50, 100, 250, 500, or 1000 mg of Fucus vesiculosus extract or any range derivable therein. In preferred embodiments, the methods comprise administering about 50 to 250 mg of Fucus vesiculosus extract.

A further aspect provides a method of decreasing oxidative stress and/or inflammation in a subject, comprising administering to the subject a sufficient amount of a composition comprising a Ribes spp. extract and/or a Hippophae rhamnoides extract to provide antioxidant and/or anti-inflammatory effects to the cells of said subject. In some embodiments, the method comprises comprising administering a Ribes nigrum and/or Ribes rubrum fruit extract. In certain embodiments, the Ribes nigrum and/or Ribes rubrum fruit extract is enriched for polyphenol compounds.

In some embodiments, the composition comprises a Hippophae rhamnoides extract and/or a Panax notoginseng extract and/or a Ribes rubrum and nigrum fruit extract and/or a Fucus vesiculosus extract. In certain embodiments, the composition comprises a Hippophae rhamnoides fruit extract or a Hippophae rhamnoides leaf extract. In some embodiments, the composition comprises a Hippophae rhamnoides leaf extract. In particular embodiments, the composition comprises a Hippophae rhamnoides fruit extract. In some embodiments, the composition comprises a Terminalia chebula fruit extract or a Terminalia chebula leaf extract. In certain embodiments, the composition comprises a Terminalia chebula fruit extract or a Terminalia chebula leaf extract comprising at least 10%, 20%, 30% or 40% tannins. In particular embodiments, the composition further comprises and Aloe extract, an Echinacea purpurea extract or an Aphanizomenon jlos aquae extract. In specific embodiments, the composition comprises an Echinacea purpurea extract comprising cichoric acid and at least 1%, 2%, 3% or 4% polyphenol content.

In some embodiments, the composition results in decreased oxidative stress in red blood cells. In certain aspects, the composition penetrates the cell membrane. In particular embodiments, the composition results in decreased reactive oxygen species (ROS) formation. In certain embodiments, administering the composition comprises oral administration.

In some embodiments, the composition is comprised in a capsule or tablet. In other embodiments, the capsule or tablet comprises an enteric coating. In some embodiments, the capsule or tablet comprises from about 25 to 50, 75, 100, 125, 150, 175, 200, 225, 250, 300, 350, 400, 450, 500, 1000, or 2000 mg of the composition or any range derivable therein. In preferred embodiments, the capsule or tablet comprises about 50 to 100, 200, 250 or 500 mg of the composition. In still further preferred embodiments, the capsule or tablet comprises about 250 to 500, 750, or 1000 mg of the composition.

In some embodiments, the methods comprise administering between 1 and 2, 3, 4, 5, 10, 25, 50, 100, 250, 500, or 1000 mg of Hippophae rhamnoides extract or any range derivable therein. In preferred embodiments, the methods comprise administering about 50 to 750 mg of Hippophae rhamnoides extract. In some embodiments, the methods comprise administering between 1 and 2, 3, 4, 5, 10, 25, 50, 100, 250, 500, or 1000 mg of Panax notoginseng extract or any range derivable therein. In preferred embodiments, the methods comprise administering about 50 to 250 mg of Panax notoginseng extract. In some embodiments, the methods comprise administering between 1 and 2, 3, 4, 5, 10, 25, 50, 100, 250, 500, or 1000 mg of Fucus vesiculosus extract or any range derivable therein. In preferred embodiments, the methods comprise administering about 50 to 250 mg of Fucus vesiculosus extract.

Another aspect provides a method of modulating immune response in a subject, comprising administering to the subject a sufficient amount of a composition comprising a Ribes spp. extract and/or a Hippophae rhamnoides extract to provide immune modulatory effects to the cells of said subject. In some embodiments, the method comprises comprising administering a Ribes nigrum and/or Ribes rubrum fruit extract. In certain embodiments, the Ribes nigrum and/or Ribes rubrum fruit extract is enriched for polyphenol compounds.

In some embodiments, the composition comprises a Hippophae rhamnoides extract and/or a Panax notoginseng extract and/or a Ribes rubrum and nigrum fruit extract and/or a Fucus vesiculosus extract. In certain embodiments, the composition comprises a Hippophae rhamnoides fruit extract or a Hippophae rhamnoides leaf extract. In some embodiments, the composition comprises a Hippophae rhamnoides leaf extract. In particular embodiments, the composition comprises a Hippophae rhamnoides fruit extract. In some embodiments, the composition comprises a Terminalia chebula fruit extract or a Terminalia chebula leaf extract. In certain embodiments, the composition comprises a Terminalia chebula fruit extract or a Terminalia chebula leaf extract comprising at least 10%, 20%, 30% or 40% tannins. In particular embodiments, the composition further comprises and Aloe extract, an Echinacea purpurea extract or an Aphanizomenon jlos aquae extract. In specific embodiments, the composition comprises an Echinacea purpurea extract comprising cichoric acid and at least 1%, 2%, 3% or 4% polyphenol content.

In certain embodiments, the composition results in activation of lymphocytes towards an effector state. In some embodiments, the composition results in an increase of CD69 expression on natural killer (NK) cells, natural killer T (NKT) cells, T cells, B lymphocytes, and/or dendritic cells as compared to CD69 expression before administration of the composition. In particular embodiments, the composition modulates immune response towards virally-infected cells or cancer cells. In some embodiments, the composition results in increased CD69 expression on T cells.

In some embodiments, the composition is comprised in a capsule or tablet. In other embodiments, the capsule or tablet comprises an enteric coating. In some embodiments, the capsule or tablet comprises from about 25 to 50, 75, 100, 125, 150, 175, 200, 225, 250, 300, 350, 400, 450, 500, 1000, or 2000 mg of the composition or any range derivable therein. In preferred embodiments, the capsule or tablet comprises about 50 to 100, 200, 250 or 500 mg of the composition. In still further preferred embodiments, the capsule or tablet comprises about 250 to 500, 750, or 1000 mg of the composition.

In some embodiments, the methods comprise administering between 1 and 2, 3, 4, 5, 10, 25, 50, 100, 250, 500, or 1000 mg of Hippophae rhamnoides extract or any range derivable therein. In preferred embodiments, the methods comprise administering about 50 to 750 mg of Hippophae rhamnoides extract. In some embodiments, the methods comprise administering between 1 and 2, 3, 4, 5, 10, 25, 50, 100, 250, 500, or 1000 mg of Panax notoginseng extract or any range derivable therein. In preferred embodiments, the methods comprise administering about 50 to 250 mg of Panax notoginseng extract. In some embodiments, the methods comprise administering between 1 and 2, 3, 4, 5, 10, 25, 50, 100, 250, 500, or 1000 mg of Fucus vesiculosus extract or any range derivable therein. In preferred embodiments, the methods comprise administering about 50 to 250 mg of Fucus vesiculosus extract.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the mobilization of endogenous stem cells in accordance with various embodiments of the present invention. Under normal physiological conditions or in response to disease or injury (A), bone marrow stem cells mobilize (C), triggered by cytokines released by the injured tissue (B), they then circulate into the bloodstream and migrate into tissues (D), where they proliferate and differentiate into cells of that tissue (E), effectuating the repair of the target tissue (F).

FIGS. 2A-2E depict a typical time course of the increase in the number of circulating stem cells in the human body after consumption of a 500 mg of 30% proanthocyanidins extract of Hippophae rhamnoides berry, in four volunteers. The increase was seen in various stem cells populations. FIG. 2A shows an average increase of 19% (P<0.05) in CD45dimCD34+ stem cells after 2 hours; FIG. 2B shows an average increase of 22% (P<0.01) in CD34+CD309− stem cells after 2 hours, FIG. 2C shows an average increase of 26.9% in CD45−CD31+CD309+ stem cells after 2 hours, and FIG. 2D shows an average increase of 29.6% (P>0.05) in very small CD34+ stem cells after 2 hours. FIG. 2E shows the maximal response at either 60 or 120 minutes, for the various types of stem cells.

FIGS. 3A & 3B show a typical time course of the decrease in the number of circulating stem cells in the human body after consumption of 500 mg of a 40% tannins extract of Terminalia chebula, in four subjects. The decrease was seen in two stem cells populations. FIG. 3A shows an average decrease of 18% in CD45dim34+ stem cells after 2 hours, and FIG. 3B shows an average decrease of 31% in CD34+CD309− stem cells after 2 hours.

FIG. 4 show the effect of a blend of 250 mg of a 30% proanthocyanidins extract of Hippophae rhamnoides berry and 250 mg of a 40% tannins extract of Terminalia chebula. Consumption of this blend leads to a increase of 41% in the number of circulating endothelial progenitor cells (CD45−CD31+CD309+) and the homing of CD45dim34+ stem cells (−10%) and CD34+CD309− stem cells (−20.7%).

FIG. 5 depicts a typical time course of the increase in the number of circulating stem cells in the human body after consumption of a 500 mg of 30% proanthocyanidins extract of Hippophae rhamnoides leaf, in three volunteers. The increase was seen in two stem cells populations. FIG. 5 shows an average increase of 58.6% (P<0.01) in Endothelial Progenitor Stem Cells (CD45⁻CD31⁺CD309⁺) after 2 hours and an average increase of 44.6% (P<0.01) in CD34⁺ small stem cells after 2 hours.

FIG. 6 depicts the proliferation of stem cells in the bone marrow and the mobilization of stem cell from the bone marrow to the blood after consumption of a 30% saponins extract of Panax notoginseng, in mice. Consumption of 500 mg of Panax notoginseng saponins (PNS) extract triggered a 68% increase in the number of bone marrow stem cells and a 67% increase in the number of circulating stem cells.

FIGS. 7A & 7B show flow cytometry profiles of blood samples showing the proportions of CD34+ lymphocytes from the peripheral blood of a human volunteer after ingestion of a 30% proanthocyanidins extract of Hippophae rhamnoides or a placebo. The X axis displays fluorescence intensity of the stem cell marker. FIG. 5B shows the corresponding number of stem cells in circulation.

FIGS. 8A & 8B show flow cytometry profiles of blood samples showing the proportions of circulating endothelial progenitor cells (CD45−CD31+CD309+) from the peripheral blood of a human volunteer after ingestion of a blend of 250 mg of a 30% proanthocyanidins extract of Hippophae rhamnoides berry and 250 mg of a 40% tannins extract of Terminalia chebula or a placebo. The X axis displays fluorescence intensity of the stem cell marker CD309, and the Y axis displays the fluorescence of the CD31 marker. FIG. 6B shows the corresponding number of CD45−CD31+CD309+ stem cells in circulation.

FIGS. 9A & 9B depict a typical time course of the decrease in the number of circulating stem cells in the human body after consumption of 500 mg of a polyphenol extract of a blend of Ribes nigrum and Ribes rubrum berry, in four volunteers. The decrease, which indicates a migration of stem cells from the blood to tissues, was seen in two stem cells populations. FIG. 9A shows an average decrease of 32.5% (P<0.01) in CD34+ stem cells and FIG. 9B shows an average decrease of 33.8% (P<0.01) in Endothelial Progenitor Stem Cells, after 2 hours.

FIGS. 10A-10C depict the antioxidant potential of Ribes nigrum and Ribes rubrum berry. FIG. 10A shows total antioxidant capacity of a polyphenol extract of a blend of Ribes nigrum and Ribes rubrum berry in Gallic Acid Equivalents (GAE) as the average f standard deviation of duplicate data points for each dose of test product. FIG. 10B depicts the percent inhibition of cellular oxidative damage as the average±standard deviation of duplicate data points for each dose of test product. FIG. 10C show the effects of formation of Reactive Oxygen Species (ROS) by human inflammatory cells when exposed to a polyphenol extract of a blend of Ribes nigrum and Ribes rubrum berry. The data is shown as the average t standard deviation of triplicate data points for each dose.

FIGS. 11A-11J depict the immune modulating effects of Ribes nigrum and Ribes rubrum berry. FIG. 11A depicts the effects on CD69 expression on NK cells. Top: The histogram shows the raw data as the average f standard deviation of triplicate data sets. Statistical significance when compared to untreated control cultures is shown when p<0.05, *, or when p<0.01, **. Bottom: The line graph shows the percent change from the untreated control cultures. FIG. 11B depicts direct effects on CD25 expression on NK cells. Top: The histogram shows the raw data as the average±standard deviation of triplicate data sets. Statistical significance when compared to untreated control cultures is shown when p<0.05, *, or when p<0.01, **. Bottom: The line graph shows the percent change from the untreated control cultures. FIG. 11C shows the direct effects on CD69 expression on NKT cells. Top: The histogram shows the raw data as the average±standard deviation of triplicate data sets. Statistical significance when compared to untreated control cultures is shown when p<0.05, *, or when p<0.01, **. Bottom: The line graph shows the percent change from the untreated control cultures. FIG. 11D shows direct effects on CD25 expression on NKT cells. Top: The histogram shows the raw data as the average±standard deviation of triplicate data sets. Statistical significance when compared to untreated control cultures is shown when p<0.05, *, or when p<0.01, **. Bottom: The line graph shows the percent change from the untreated control cultures. FIG. 11E shows direct effects on CD69 expression on T cells. Top: The histogram shows the raw data as the average f standard deviation of triplicate data sets. Statistical significance when compared to untreated control cultures is shown when p<0.05, *, or when p<0.01, **. Bottom: The line graph shows the percent change from the untreated control cultures. FIG. 11F shows direct effects on CD25 expression on T cells. Top: The histogram shows the raw data as the average±standard deviation of triplicate data sets. Statistical significance when compared to untreated control cultures is shown when p<0.05, *, or when p<0.01, **. Bottom: The line graph shows the percent change from the untreated control cultures. FIG. 11G shows direct effects on CD69 expression on non-NK non-T cells. Top: The histogram shows the raw data as the average f standard deviation of triplicate data sets. Statistical significance when compared to untreated control cultures is shown when p<0.05, *, or when p<0.01, **. Bottom: The line graph shows the percent change from the untreated control cultures. FIG. 11H shows direct effects on CD25 expression on nonNK nonT cells. Top: The histogram shows the raw data as the average±standard deviation of triplicate data sets. Statistical significance when compared to untreated control cultures is shown when p<0.05, *, or when p<0.01, **. Bottom: The line graph shows the percent change from the untreated control cultures. FIG. 11I direct effects on CD69 expression on monocytes. Top: The histogram shows the raw data as the average f standard deviation of triplicate data sets. Statistical significance when compared to untreated control cultures is shown when p<0.05, *, or when p<0.01, **. Bottom: The line graph shows the percent change from the untreated control cultures. FIG. 11J: direct effects on CD25 expression on monocytes. Top: The histogram shows the raw data as the average f standard deviation of triplicate data sets. Statistical significance when compared to untreated control cultures is shown when p<0.05, *, or when p<0.01, **. Bottom: The line graph shows the percent change from the untreated control cultures.

FIGS. 12A-12J depict anti-inflammatory effects of Ribes nigrum and Ribes rubrum berry. FIG. 12A depicts inflammatory effects on CD69 expression on NK cells. Top: The histogram shows the raw data as the average f standard deviation of triplicate data sets. Statistical significance when compared to LPS control cultures is shown when p<0.05, *, or when p<0.01, **. Bottom: The line graph shows the percent change from the LPS control cultures. FIG. 12B depicts inflammatory effects on CD25 expression on NK cells. Top: The histogram shows the raw data as the average f standard deviation of triplicate data sets. Statistical significance when compared to LPS control cultures is shown when p<0.05, *, or when p<0.01, **. Bottom: The line graph shows the percent change from the LPS control cultures. FIG. 12C depicts inflammatory effects on CD69 expression on NKT cells. Top: The histogram shows the raw data as the average±standard deviation of triplicate data sets. Statistical significance when compared to LPS control cultures is shown when p<0.05, *, or when p<0.01, **. Bottom: The line graph shows the percent change from the LPS control cultures. FIG. 12D depicts inflammatory effects on CD25 expression on NKT cells. Top: The histogram shows the raw data as the average f standard deviation of triplicate data sets. Statistical significance when compared to LPS control cultures is shown when p<0.05, *, or when p<0.01, **. Bottom: The line graph shows the percent change from the LPS control cultures. FIG. 12E depicts inflammatory effects on CD69 expression on T cells. Top: The histogram shows the raw data as the average f standard deviation of triplicate data sets. Statistical significance when compared to LPS control cultures is shown when p<0.05, *, or when p<0.01, **. Bottom: The line graph shows the percent change from the LPS control cultures. FIG. 12F depicts inflammatory effects on CD25 expression on T cells. Top: The histogram shows the raw data as the average±standard deviation of triplicate data sets. Statistical significance when compared to LPS control cultures is shown when p<0.05, *, or when p<0.01, **. Bottom: The line graph shows the percent change from the LPS control cultures. FIG. 12G depicts inflammatory effects on CD69 expression on non-NK non-T cells. Top: The histogram shows the raw data as the average±standard deviation of triplicate data sets. Statistical significance when compared to LPS control cultures is shown when p<0.05, *, or when p<0.01, **. Bottom: The line graph shows the percent change from the LPS control cultures. FIG. 12H depicts inflammatory effects on CD25 expression on non-NK non-T cells. Top: The histogram shows the raw data as the average±standard deviation of triplicate data sets. Statistical significance when compared to LPS control cultures is shown when p<0.05, *, or when p<0.01, **. Bottom: The line graph shows the percent change from the LPS control cultures. FIG. 12I depicts inflammatory effects on CD69 expression on monocytes. Top: The histogram shows the raw data as the average f standard deviation of triplicate data sets. Statistical significance when compared to LPS control cultures is shown when p<0.05, *, or when p<0.01, **. Bottom: The line graph shows the percent change from the LPS control cultures. FIG. 12J depicts inflammatory effects on CD25 expression on monocytes. Top: The histogram shows the raw data as the average f standard deviation of triplicate data sets. Statistical significance when compared to LPS control cultures is shown when p<0.05, *, or when p<0.01, **. Bottom: The line graph shows the percent change from the LPS control cultures.

FIGS. 13A-13J depict immune modulatory effects of Ribes nigrum and Ribes rubrum berry in the context of a viral mimetic insult. FIG. 13A depicts effects on CD69 expression on NK cells, in context of a viral mimetic challenge. Top: The histogram shows the raw data as the average±standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge. FIG. 13B depicts effects on CD25 expression on NK cells, in context of a viral mimetic challenge. Top: The histogram shows the raw data as the average±standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge. FIG. 13C depicts effects on CD69 expression on NKT cells, in context of a viral mimetic challenge. Top: The histogram shows the raw data as the average f standard deviation of triplicate data sets. Statistical significance when compared to Poly I:C-induced anti-viral challenge is shown when p<0.05, or when p<0.01, **. Bottom: The line graph shows the percent change from the Poly I:C-induced anti-viral challenge. FIG. 13D depicts effects on CD25 expression on NKT cells, in context of a viral mimetic challenge. Top: The histogram shows the raw data as the average±standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge. FIG. 13E depicts effects on CD69 expression on T cells, in context of a viral mimetic challenge. Top: The histogram shows the raw data as the average f standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge. FIG. 13F depicts effects on CD25 expression on T cells, in context of a viral mimetic challenge. Top: The histogram shows the raw data as the average±standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge. FIG. 13G depicts effects on CD69 expression on non-NK non-T cells, in context of a viral mimetic challenge. Top: The histogram shows the raw data as the average±standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge. FIG. 13H depicts effects on CD25 expression on non-NK non-T cells, in context of a viral mimetic challenge. Top: The histogram shows the raw data as the average f standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge. FIG. 13I depicts effects on CD69 expression on monocytes, in context of a viral mimetic challenge. Top: The histogram shows the raw data as the average±standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge. FIG. 13J depicts effects on CD69 expression on monocytes, in context of a viral mimetic challenge. Top: The histogram shows the raw data as the average f standard deviation of triplicate data sets. Statistical significance when compared to Poly I:C-induced anti-viral challenge is shown when p<0.05, *, or when p<0.01, **. Bottom: The line graph shows the percent change from the Poly I:C-induced anti-viral challenge.

### DESCRIPTION OF ILLUSTRATIVE EMBODIMENTS

### I. The Present Embodiments

Increased presence of stem cells in the blood can provide many beneficial effects. For example, it can boost and better regulate the immune system. However, to date limited supplements and nutraceuticals have been evaluated for the ability to provide enhanced stem cell mobilization. Studies herein, however, demonstrate that extracts from Hippophae rhamnoides, Terminalia chebula, Panax notoginseng and Ribes spp. enhance stem cell mobilization in human subjects. Accordingly, nutraceutical compositions comprising these extracts are provided herein and can be used to enhance stem cell mobilization and the general well-being of subjects.

### II. Definitions

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Differentiation” as used herein refers to the process by which cells become more specialized to perform biological functions. For example, hematopoietic stem cells, hematopoietic progenitors and/or stem cells may change from multipotent stem cells into cells committed to a specific lineage and/or cells having characteristic functions, such as mature somatic cells. Differentiation is a property that is often totally or partially lost by cells that have undergone malignant transformation.

“Enhancement,” “enhance” or “enhancing” as used herein refers to an improvement in the performance of or other physiologically beneficial increase in a particular parameter of a cell or organism. At times, enhancement of a phenomenon is quantified as a decrease in the measurements of a specific parameter. For example, migration of stem cells may be measured as a reduction in the number of stem cells circulating in the circulatory system, but this nonetheless may represent an enhancement in the migration of these cells to areas of the body where they may perform or facilitate a beneficial physiologic result, including, but not limited to, differentiating into cells that replace or correct lost or damaged function. In one embodiment, enhancement refers to a 15%, 20%, 30% or greater than 50% reduction in the number of circulating stem cells. In one specific, non-limiting example, enhancement of stem cell migration may result in or be measured by a decrease in a population of the cells of a non-hematopoietic lineage, such as a 15%, 20%, 30%, 50%, 75% or greater decrease in the population of cells or the response of the population of cells. In one embodiment, an enhanced parameter is the trafficking of stem cells. In one embodiment, the enhanced parameter is the release of stem cells from a tissue of origin. In one embodiment, an enhanced parameter is the migration of stem cells. In another embodiment, the parameter is the differentiation of stem cells. In yet another embodiment, the parameter is the homing of stem cells.

“Fucus vesiculosus” as used herein, also known as Bladderwrack or kelp, refers to a seaweed found on the coasts of the North Sea, the western Baltic Sea, and the Atlantic and pacific Oceans.

“Hematopoietic agent” as used herein refers to a compound, antibody, nucleic acid molecule, protein, cell or other molecule that affects hematopoiesis. A molecular agent can be a naturally-occurring molecule or a synthetic molecule. In some instances, the agent affects the growth, proliferation, maturation, migration or differentiation or release of hematopoietic cells.

“Hematopoietic stem cells” as used in the present invention means multipotent stem cells that are capable of eventually differentiating into all blood cells including, erythrocytes, leukocytes, megakaryocytes, and platelets. This may involve an intermediate stage of differentiation into progenitor cells or blast cells. The term “hematopoietic progenitors”, “progenitor cells” or “blast cells” are used interchangeably in the present invention and describe maturing HSCs with reduced differentiation potential, but are still capable of maturing into different cells of a specific lineage, such as myeloid or lymphoid lineage. “Hematopoietic progenitors” include erythroid burst forming units, granulocyte, erythroid, macrophage, megakaryocyte colony forming units, granulocyte, erythroid, macrophage, and granulocyte macrophage colony-forming units.

“Hippophae rhamnoides” as used herein, also known as sea buckthorn, refers to a species of fruit-bearing flowering plant in the family Elaeagnaceae, native to the cold-temperate regions of Europe and Asia.

“Homing” as used herein refers to the process of a cell migrating from the circulatory system into a tissue or organ. In some instances, homing is accomplished via tissue-specific adhesion molecules and adhesion processes. Homing may refer to the migration back to the bone marrow.

“Isolated biological component” (such as a nucleic acid molecule, polypeptide, polysaccharide or other biological molecule) as used herein refers to a biological component that has been substantially separated or purified away from other biological components in which the component naturally occurs. Nucleic acids and proteins may be isolated by standard purification methods, recombinant expression in a host cell, or chemically synthesized.

“Modulation” or “modulates” or “modulating” as used herein refers to upregulation (i.e., activation or stimulation), down regulation (i.e., inhibition or suppression) of a response or the two in combination or apart.

“Migration” as used herein refers to the central process for movement of cells in the development and maintenance of multicellular organisms. Cells often migrate in response to, and towards, specific external signals, commonly referred to as chemotaxis. Migration includes the process of a cell moving from the circulatory system into a tissue or organ. More specifically, circulating stem cells are tethered to the surface of capillary endothelium via expression of adhesion molecules of cell surfaces, resulting in cytoskeletal changes in both endothelium and stem cells, and allowing movement through the capillary wall en route to a tissue and/or organ site. In some instances, homing is accomplished via tissue-specific adhesion molecules and adhesion processes.

“Panax notoginseng” as used herein, also known as Chinese ginseng or notoginseng or Asian ginseng, refers to a species of plant in the family Araliaceae, native to China.

“Pharmaceutically acceptable carriers” as used herein refer to conventional pharmaceutically acceptable carriers useful in this invention.

“Polysaccharide” as used herein refers to a polymer of more than about ten monosaccharide residues linked glycosidically in branched or unbranched chains.

As used herein, the term “polyphenol” is used to describe a compound or mixture of compounds from a natural source which contains two or more phenol groups and is devoid of nitrogen containing groups. In particular, the polyphenol may satisfy the criteria of the Quideau definition of a polyphenol. In some embodiments, the polyphenol is moderately water-soluble. In some embodiments, the polyphenol is a compound with a molecular weight from about 500 to about 4000 Da. The polyphenol may be a compound containing 2, 4, 6, 8, 10, or more than 12 phenolic hydroxylic groups. The polyphenol may contain from about 5 to about 7 aromatic rings per 1000 Da of molecular weight. The polyphenol may be a compound derived either from the shikimate or phenylpropanoid skeleton and/or made from the polyketide pathway. In some embodiments, the polyphenol is a monomer which may polymerize to form a compound with the above characteristics. In some embodiments, the polyphenol satisfies the criteria of the White-Bate-Smith-Swain-Haslam (WBSSH) definition of a polyphenol compound.

As used herein, the term “proanthocyanidin” is used to describe a compound or mixture of compounds from a natural source which results from the polymerization (or oligomerization) of two or more flavonoids. In particular, the oligomerization may be an oligomer of catechin or epicatechin and esters of them. In some embodiments, the term proanthocyanidin includes complexes of multiple polyphenolic building blocks and may be known as tannins. In some embodiments, the term “proanthocyanidin” refers to dimers or trimers of catechins. “Progenitor cell” as used herein refers to a cell that gives rise to progeny in a defined cell lineage.

“Recruitment” of a stem cell as used herein refers to a process whereby a stem cell in the circulatory system migrates into specific site within a tissue or organ. Recruitment may be facilitated by a compound or molecule, such as a chemoattractant signal or cell receptor. For example, both CXCR4 and SDF-1 have identified roles in stem cell homing and migration.

“Releasing agent” as used herein are mobilization agents capable of promoting the release and egress of stem cells from a tissue of origin. Release of stem cells from a tissue of origin may be demonstrated, for example, by an increase in circulating stem cells in the circulatory or immune system, or by the expression of markers related to egress of stem cells from a tissue of origin, such as bone marrow. For example, a releasing agent increases the number of bone marrow-derived stem cells and/or hematopoietic stem cells in the peripheral blood. In another embodiment, the releasing agent affects the number of stem cells, such as CD34.sup.high (CD34+) cells, circulating in the peripheral blood.

“Ribes spp.” or “Ribes nigrum & Ribes rubrum” as used herein, also known as black and red currant, refers to a member of the genus Ribes in the gooseberry family Grossulariaceae. Though cultivated in various parts of the world, these berries are native to many parts of Europe and Asia.

“Stem cells” as used herein are cells that are not terminally differentiated and are therefore able to produce cells of other types. Characteristic of stem cells is the potential to develop into mature cells that have particular shapes and specialized functions, such as heart cells, skin cells, or nerve cells. Stem cells are divided into three types, including totipotent, pluripotent, and multipotent. “Totipotent stem cells” can grow and differentiate into any cell in the body and thus, can form the cells and tissues of an entire organism. “Pluripotent stem cells” are capable of self-renewal and differentiation into more than one cell or tissue type. “Multipotent stem cells” are clonal cells that are capable of self-renewal, as well as differentiation into adult cell or tissue types. Multipotent stem cell differentiation may involve an intermediate stage of differentiation into progenitor cells or blast cells of reduced differentiation potential, but are still capable of maturing into different cells of a specific lineage. The term “stem cells”, as used herein, refers to pluripotent stem cells and multipotent stem cells capable of self-renewal and differentiation. “Bone marrow-derived stem cells” are the most primitive stem cells found in the bone marrow which can reconstitute the hematopoietic system, possess endothelial, mesenchymal, and pluripotent capabilities. Stem cells may reside in the bone marrow, either as an adherent stromal cell type, or as a more differentiated cell that expresses CD34, either on the cell surface or in a manner where the cell is negative for cell surface CD34. “Adult stem cells” are a population of stem cells found in adult organisms with some potential for self-renewal and are capable of differentiation into multiple cell types. Other examples of stem cells are marrow stromal cells (MSCs), HSC, multipotent adult progenitor cells (MAPCs), very small embryonic-like stem cells (VSEL), epiblast-like stem cell (ELSC) or blastomere-like stem cell (BLSC).

“Stem cell circulation agent” (SCCA), “mobilization agent”, and/or “mobilization factor” as used herein refers to one or more compounds, antibodies, nucleic acid molecules, proteins, polysaccharides, cells, or other molecules, including, but not limited to, neuropeptides and other signaling molecules, that affects the release, circulation, homing and/or migration of stem cells from the circulatory system into tissue or organ. A molecular agent may be a naturally occurring molecule or a synthetic molecule. Examples of mobilization agents include “releasing agents”, wherein a releasing agent is capable of promoting the egress of stem cells from a tissue of origin and also “migration agents”, wherein a migration agent is capable of promoting the process of a cell moving from the circulatory system into a tissue or organ.

“Subject” as used herein includes all animals, including mammals and other animals, including, but not limited to, companion animals, farm animals and zoo animals. The term “animal” can include any living multi-cellular vertebrate organisms, a category of which non-limiting examples include, a mammal, a bird, a simian, a dog, a cat, a horse, a cow, a rodent, and the like. Likewise, the term “mammal” includes both human and non-human mammals.

“Terminalia chebula” as used herein, also known as black or chebulic myrobalan, refers to a species of Terminalia that is native to South Asia. Both the fruit and the leaves can be used in the present invention. Terminalia chebula can refer to various varieties, such as Terminalia “chebula chebula” and Terminalia “chebula tomentella”, as well as various Terminalis species such as “bellerica”, “catappa”, “arjuna”, “albida”, “elliptica”, and “porphyrocarpa”.

“Therapeutically effective amount” as used herein refers to the quantity of a specified composition, or active agent in the composition, sufficient to achieve a desired effect in a subject being treated. For example, this can be the amount effective for enhancing migration of stem cells that replenish, repair, or rejuvenate tissue. In another embodiment, a “therapeutically effective amount” is an amount effective for enhancing trafficking of stem cells, such as increasing release of stem cells, as can be demonstrated by elevated levels of circulating stem cells in the bloodstream. In still another embodiment, the “therapeutically effective amount” is an amount effective for enhancing homing and migration of stem cells from the circulatory system to various tissues or organs, as can be demonstrated be decreased level of circulating stem cells in the bloodstream and/or expression of surface markers related to homing and migration. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Trafficking” as used herein refers to the process of movement of a cell from the tissue of origin, traveling within the circulatory or immune system, and localization towards a site within a tissue and/or organ. Trafficking also includes stem cell mobilization, beginning with release from a tissue of origin, such as egress of stem cells from bone marrow. Trafficking further includes movement of a cell from the tissue of origin, homing by adhesion to the endothelium, transmigration, and final migration within the target tissue and/or organ. Furthermore, trafficking may include the process of movement of a cell of the immune system. One specific, non-limiting example of trafficking is the movement of a stem cell to a target organ, also referred to as migration. Another specific, non-limiting example of trafficking is the movement of a B-cell or a pre-B-cell leaving the bone marrow and moving to a target organ.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

As described, stem cells are unique cells that possess the capacity to differentiate into more specialized cells. One particular type of stem cell, hematopoietic stem cells (HSCs), are capable of differentiating into many different types of blood cells. In addition, HSCs typically reside in the bone marrow, where proliferation and self-renewal of the cells allows HSCs to be involved in the support and maintenance of the hematopoietic system. Existing scientific literature has chiefly focused on HSCs' potential to develop into hematopoietic lineage cells derivatives. Emerging evidence has further identified the capacity for HSCs to also differentiate into non-hematopoietic, tissue specific cells. Recently, HSCs have been found to possess the capacity to differentiate into a variety of tissue-specific cell types, such as myocytes, hepatocytes, osteocytes, glial cells, and neurons. As a result, aside from forming blood and immune cells, HSCs are responsible for constant maintenance and repair of virtually every tissue and organ of the body.

Similarly, bone marrow stem cells (BMSCs) were recently shown to have significant capability to become cells of other tissues. In the bone marrow, stem cells duplicate using a process known as “asymmetrical cellular division” according to which the two daughter cells are not identical; one cell retains the original DNA and remains in the bone marrow whereas the other cell contains the DNA copies and is released in the blood where it migrates into various tissues in need of repair. BMSCs have been traditionally considered to have little potential for plasticity, being limited in their development to red blood cells, lymphocytes, platelets, bone and connective tissue. However, much scientific work has been published over the past few years that demonstrates the exceptional plasticity of BMSC. For example, after transplantation, BMSCs and HSCs were shown to have the ability to become muscle cells, heart cells, endothelium capillary cells, liver cells, as well as lung, gut, skin, and brain cells. As a further illustrative example, some studies report the ability of HSC to become liver cells upon contact with specific liver-derived molecules, but this process took place within hours. Briefly, HSCs were co-cultured with either normal or damaged liver tissue separated by a semi-permeable membrane (pores large enough to let molecules pass through, but small enough to prevent the passage of cells from one compartment to the other, pore size 0.4 μm). Using immunofluorescence assay methods to detect molecules specific for either HSCs (CD45) or liver cells (albumin), the researchers could follow the transformation of the population of cells placed in the upper compartment. When HSCs were cultured alone for 8 hours, they only expressed CD45 and no albumin, indicating that no HSCs had differentiated into liver cells. However, when HSCs were exposed to injured liver tissue, they rapidly became positive for albumin. Over time, the population of cells positive for CD45 began to decrease as the population positive for albumin began to increase. Albumin-positive cells were seen as early as 8 hours into the procedure and increased in frequency to 3.0% at 48 hours. The conversion was minimal and delayed when HSCs were exposed to undamaged liver (control for injury).

Because HSCs and BMSCs play an important role in the healing and regenerative processes of various tissues and organs in the body beyond their traditional role in maintaining hematopoietic and immune systems of the body, activation and enhancement of stem cell trafficking may amplify these physiological processes and provide a potential therapy for various pathologies. The classic source of HSCs and BMSCs is bone marrow, which includes hip, ribs, sternum and other bone structures. Bone provides a unique regulatory microenvironment for HSCs and BMSCs, which comprises mesenchymal stem cells, including interaction with specific extracellular matrix glycoproteins and a uniquely rich mineral signature. This stem cell “niche” contains a great deal of critical molecular interactions which guide the response of stem cells to specific physiological conditions. The niche may be an important focal point for changes in the state of tissue that result in a change in the regenerative processes rooted in stem cell activity. (Adams and Scadden, 2006)

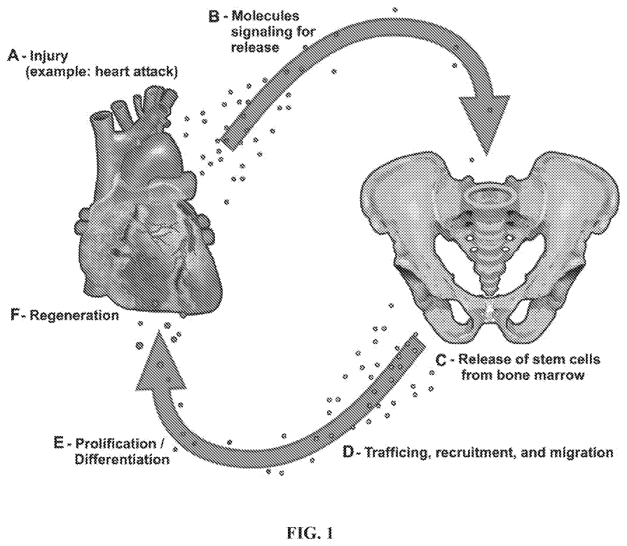

Beyond populations of stem cells found in bone marrow, stem cells are also present in the peripheral bloodstream of normal, healthy persons. It has been known for decades that a small number of stem and progenitor cells circulate in the bloodstream, but more recent studies have shown that greater numbers of stem cells can be coaxed into mobilization from marrow to blood by injecting the donor with a cytokine, such as granulocyte-colony stimulating factor (G-CSF). Despite this advance, the natural process by which stem cells are released from bone marrow and migrate towards a site within tissue and/or an organ is not fully understood. A leading model involves the chemokine, Stromal-Derived Factor-1 (SDF-1) and its specific receptor, CXCR4. In this capacity, the binding of SDF-1 to CXCR4 leads to adherence of stem cells to bone marrow through increased expression of adhesion molecules on the cell membrane surface. Disruption of adhesion of stem cells to the bone marrow matrix thus promotes mobilization of stem cells into the peripheral bloodstream. (FIG. 1C) Some factors such as G-CSF or IL-8 may interfere with adhesion through elevated activation of proteolytic enzymes or degradation of the SDF-1 ligand. Other types of molecules, such as L-selectin blockers, may instead down-regulate CXCR4 expression which in turn reduces stem cell adhesion to the bone marrow environment. Without wishing to be bound by any theory, enhancing binding of SDF-1 to CXCR4 promote adherence, therefore L-selectin blockers such as sulfated fucans, which reduces CXCR4 expression, can trigger stem cell mobilization.

Stem cells circulating in the peripheral bloodstream are recruited to sites of tissue in need of repair and regeneration through homing and extravasation. This mobilization of stem cells into the bloodstream and subsequent migration to the site of tissue injury results from a combination of mechanical and chemoattractant signals. Mechanical force or other factors may activate L-selectins on the surface of stem cells. Activation of L-selectins, in turn, may promote elevated expression of the receptor, CXCR4. Cells at the site of tissue injury may also secrete SDF-1 ligand, thereby attracting stem cells expressing receptor CXCR4 to the injury site. The interaction of SDF-1 and CXCR4 promotes sufficient adhesion to halt circulation of a stem cell in the peripheral blood stream. (FIG. 1B) Based on this model, L-selectin blockers such as sulfated fucans, may possess a critical capacity to mobilize stem cells into the bloodstream, with subsequent homing, extravasation and migration into tissue promoting regenerative maintenance and repair of cells and tissues in an organism. Whereas G-CSF is released from injured tissue and its presence in the bloodstream triggers stem cell release from bone marrow, dietary supplements composed of L-selectin blockers may possibly support the phenomenon of natural regeneration and repair in the body.

### III. Extracts and Compositions of the Embodiments

Exemplary Hippophae rhamnoides Extract

Various methods for the extraction from fruits and berries are envisioned to be used according to the embodiments. In one example, Hippophae rhamnoides berries could be to subject to cold extraction with 4 L of 80% methanol (v/v; 24 h). This method could further comprise ultrasonic treatment (2 Å˜10 min). The plant material can be further extracted with 80% methanol (v/v; 4 L; 1 h), under reflux. Both extracts can be combined, filtered, and concentrated in a rotary evaporator to remove the organic solvent. The residue can be applied onto a short LiChroprep 40-63 μm RP-18 column (Millipore Corp., Bedford, Mass.) and equilibrated with water. The column can be washed with water to remove highly polar extract constituents, and bound phenolic compounds can be eluted with 50% methanol (v/v). The obtained 50% methanol eluate can be concentrated by a rotary evaporation and subsequently dried, to yield a dry phenolic fraction. In another example, a similar method is used to extract polyphenols from the leaves of Hippophae rhamnoides.

Exemplary Terminalia chebula Extract

Terminalia chebula, also known as Haritaki in Ayurvedic Medicine and the “King of plant” in Tibetan medicine, has been used for centuries in India and Tibet for the treatment of various ailments touching various aspects of human health. While the fruit of Terminalia chebula has been used historically to treat problems of the digestive system, ulcers, colitis, as well as to improve memory and brain function, studies have documented it can reverse hyperglycemia and even diabetes, accelerate wound healing, alleviate symptoms associated with Alzheimer's disease, neuropathy, and reduce inflammation. However, a mechanism of action was never proposed that could explain all these various benefits touching various aspects of human health.

In Terminalia chebula, up to 50% of the total phytoconstituents are hydrolysable tannins. These tannins contain phenolic carboxylic acid like gallic acid, ellagic acid, chebulic acid and gallotannins. Ellagitannin such as punacalagin, casurarinin, corilagin and terchebulin and others such as chebulanin, neochebulinic acid, chebulagic acid and chebulinic acid were reported in literature. The tannin content varies with the geological variation. Flavonol glycosides, triterpenoids, coumarin conjugated with gallic acid called chebulin, as well as phenolic compounds have also been isolated.

Various methods could be used for the extraction of phytoconstituents from Terminalia chebula. In one example method dried fruits of T. chebula could be to pulverize in a blender, and then to suspend the resultant powered fruits in 20 mL of an extraction solvent: for example 100% ethanol, 80% ethanol (ethanol/water=8:2, v/v) or 50% ethanol (ethanol/water=1:1, v/v). Extractions can be carried out at room temperature for 36 h with continuous stirring and may then be treated with ultrasonication for 1 h. After filtration, the solvent from this extract can be evaporated and the residues can be dried using various methods.

Exemplary Panax notoginseng Extract

Panax notoginseng is a species of the genus Panax and is commonly referred to in English as Chinese ginseng or simply notoginseng. It Chinese name means “cure-all”. It is endemic to China where it is referred to as three-seven ginseng, referring to the time it takes (3-7 years) for the roots to mature prior to harvest. notoginseng is a unique source of ginsenosides, which are glycosylated triterpenes also known as saponins. These compounds have been reported to have many health-promoting properties, namely antioxidants, anti-inflammation, cardioprotective, anticancer, and general anti-aging.

Various methods could be used for the extraction of phytoconstituents, more specifically saponins and ginsenosides, from Panax notoginseng. In one example method, notoginseng roots are ground into a powder and extracted in a solution of water and ethanol at either 50/50 or 80/20 w/EtOH, at 80-90° C. for 20-24 h. To enhance the extraction process, the method of heat-reflux can be used using a Soxhlet with shaking or ultrasound. The solution is then filtered and dried.

Exemplary Ribes Spp. Extract

Ribes is a genus of about 150 known species of flowering plants native throughout the temperate regions of the Northern Hemisphere. Specifically, two species of Ribes carry fruits that have been known and documented to have strong health-promoting properties: Ribes nigrum and Ribes rubrum, commonly known as blackcurrant and redcurrant. Both berries are in the gooseberry family and are endemic to Northern Europe and Northern Asia. The berries are rich in vitamin C and in polyphenols, more specifically anthocyanins, flavanols, flavonols and resveratrol. All these compounds have been documented for their antioxidant properties as well as other health-promoting properties, similar to Hippophae rhamnoides. Extraction of polyphenols from Ribes spp. follows a procedure similar to extraction of polyphenols from Hippophae berries.

Embodiments of the present invention provides new compositions and methods for providing a wide range of clinical and physiological benefits to a subject in need thereof by the administration of a mobilization agent. While not wishing to be bound by any particular theory, the inventors believe that the beneficial and other physiological results obtained through administration of the inventive compositions result from enhancing stem cell mobilization and trafficking that follows the administration of the mobilization agent.

Described herein are compositions including a mobilization agent with one or more components selected from the group including: Hippophae rhamnoides or extracts thereof, Terminalia chebula or extracts thereof, Ribes spp. of extract thereof, and/or Panax notoginseng of extract thereof. The mobilization agents may be combined together in one or more compositions or they may be administered or consumed separately. They may have individual physiological effects, additive effects and/or synergistic effects with one another. In some embodiments, the mobilization agent is capable of functioning as a releasing agent, promoting the release and egress of stem cells from a tissue of origin. In various embodiments, the composition is a pharmaceutical composition including the above components and a pharmaceutically acceptable carrier.

In one embodiment, a mobilization agent is administered to a subject, for example Hippophae rhamnoides, though the subject may be provided a mixture of Hippophae rhamnoides and other mobilization agents. In some embodiments, the subject consumes and digests whole Hippophae rhamnoides berries. The fruits may be fresh, frozen, freeze-dried, dehydrated, or preserved in some other manner. Therefore, Hippophae rhamnoides, as described herein, encompasses both whole fruits and extracts thereof. In another embodiment, the subject consumes Hippophae rhamnoides leaves of extract thereof. In one embodiment, the mobilization agent is an extract of Hippophae rhamnoides, or an isolated component or compound extracted from Hippophae rhamnoides, such as a compound found in a polysaccharide-rich fraction of Hippophae rhamnoides extract or a polyphenol-rich fraction of Hippophae rhamnoides extract. In one embodiment, the mobilization agent is a proanthocyanidin extract of Hippophae rhamnoides. Hippophae rhamnoides can be provided alone as an isolated or purified substance, or may be part of a composition including a pharmaceutically acceptable carrier.

In one embodiment, a mobilization agent is administered to a subject, for example Terminalia chebula, though the subject may be provided a mixture of Terminalia chebula and other mobilization agents. In some embodiments, the subject consumes and digests whole Terminalia chebula fruits. The fruits may be fresh, frozen, freeze-dried, dehydrated, or preserved in some other manner. Therefore, Terminalia chebula, as described herein, encompasses both whole fruit, leaves, and extracts thereof. In one embodiment, the mobilization agent is an extract of Terminalia chebula, or an isolated component or compound extracted from Terminalia chebula, such as a compound found in a tannin-rich fraction of Terminalia chebula extract or an extract concentrating chebulic acid. Terminalia chebula can be provided alone as an isolated or purified substance, or may be part of a composition including a pharmaceutically acceptable carrier.

In one embodiment, a mobilization agent is administered to a subject, for example Ribes spp. fruits, though the subject may be provided a mixture of Ribes nigrum and/or Ribes rubrum and other mobilization agents. In some embodiments, the subject consumes and digests whole Ribes spp. fruits. The fruits may be fresh, frozen, freeze-dried, dehydrated, or preserved in some other manner. Therefore, Ribes spp., as described herein, encompasses both whole fruit, leaves, and extracts thereof. In one embodiment, the mobilization agent is an extract of Ribes spp., or an isolated component or compound extracted from Ribes spp., such as a compound found in a polyphenol-rich fraction of Ribes spp. extract or an extract concentrating anthocyanins. Ribes spp. can be provided alone as an isolated or purified substance, or may be part of a composition including a pharmaceutically acceptable carrier.

In one embodiment, a mobilization agent is administered to a subject, for example Panax notoginseng root, though the subject may be provided a mixture of Panax notoginseng and other mobilization agents. In some embodiments, the subject consumes and digests whole Panax notoginseng root. The roots may be fresh, frozen, freeze-dried, dehydrated, or preserved in some other manner. Therefore, Panax notoginseng, as described herein, encompasses both whole fruit, leaves, roots and extracts thereof. In one embodiment, the mobilization agent is an extract of Panax notoginseng, or an isolated component or compound extracted from Panax notoginseng, such as a compound found in a saponins fraction of Panar notoginseng extract or an extract concentrating ginsenosides. Panar notoginseng can be provided alone as an isolated or purified substance, or may be part of a composition including a pharmaceutically acceptable carrier.

In various embodiments, the dosage of each of the one or more mobilization agents in the composition can include 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of the mobilization agents. For example, the one or more mobilization agents in the compositions can be combined at each of these variable dosage amounts. For example, a representative set of dosages in the composition are shown in Table 1. In various embodiments, the composition includes 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of Hippophae rhamnoides or extracts thereof, Terminalia chebula or extracts thereof, Ribes spp. of extract thereof, and/or Panax notoginseng of extract thereof. In various embodiments, the dosages can contain one or more mobilization agents for a total amount of 50-250, 250-500, 500-750, 750-1000, 1000-2000, 2000-3000, 3000 mg or more. For example, in various embodiments, the pharmaceutical composition includes 750 mg or less of Hippophae rhamnoides or extracts thereof and 1000 mg or less of Terminalia chebula or extracts thereof. In other embodiments, the pharmaceutical composition includes 750 mg or less of Ribes spp. or extracts thereof and 500 mg or less of Panax notoginseng or extracts thereof. In various embodiments, the total dosage amount is administered daily for one or more days, or multiple times in a single day.

The present invention further provides a method of enhancing the trafficking of stem cells in a subject. In one embodiment, the level of trafficking of stem cells relates to the number of circulating hematopoietic stem cells (HSCs) in the peripheral blood of a subject. In another embodiment, the level of trafficking of stem cells relates to the number of circulating bone marrow-derived stem cells in the peripheral blood of a subject. In various embodiments, enhancing the trafficking of stem cells in a subject, includes administering a therapeutically effective amount of a mobilization agent, thereby increasing the release, circulation, homing and/or migration of stem cells in the subject, regardless of the route of administration.

In another embodiment, the method provided herein enhances the trafficking of stem cells in a subject, including administering a therapeutically effective amount of a composition containing one or more of the following components selected from the group including: Hippophae rhamnoides or extracts thereof, Terminalia chebula or extracts thereof, Ribes spp. of extract thereof, and/or Panax notoginseng of extract thereof, thereby enhancing the trafficking of stem cells in the subject. In one embodiment, enhancement of stem cell trafficking may be measured by assaying the response of stem cells to a particular dose of a composition containing one or more of the following components selected from the group including: Hippophae rhamnoides or extracts thereof, Terminalia chebula or extracts thereof, Ribes spp. of extract thereof, and/or Panax notoginseng of extract thereof, thereby enhancing the trafficking of stem cells in the subject.

In another embodiment, a method of enhancing the trafficking of stem cells in a subject includes a transient increase in the population of circulating stem cells, such as stem cells following administration of a mobilization agent. In one embodiment, the stem cells are hematopoietic stem cells (HSCs). In another embodiment, the stem cells are bone marrow-derived stem cells. In various embodiments, the stem cells are CD45, CD34⁺, CD34⁺, CD34⁺ KDR⁻, or CD45−CD31⁺KDR⁺, CD34⁺CD133⁻, CD34⁺CD133⁺, or express various sub-combinations of these markers. In one embodiment, providing a mobilization agent to a subject will enhance release of that subject's stem cells within a certain time period, such as less than 12 days, less than 6 days, less than 3 days, less than 2, or less than 1 days. In an alternative embodiment, the time period is less than 12 hours, 6 hours, less than about 4 hours, less than about 2 hours, or less than about 1 hour following administration. In various embodiments, release of stem cells into the circulation from about 1, 2, or 3 hours following administration. In another embodiment, released stem cells enter the circulatory system and increase the number of circulating stem cells within the subject's body. In another embodiment, the percentage increase in the number of circulating stem cells compared to a normal baseline may be about 25%, about 50%, about 100% or greater than about 100% increase as compared to a control. In one embodiment, the control is a base line value from the same subject. In another embodiment, the control is the number of circulating stem cells in an untreated subject, or in a subject treated with a placebo or a pharmacological carrier.

In various embodiments, administering a therapeutically effective amount of a composition includes oral administration of a dosage containing one or more mobilization agents in the amount of 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of the mobilization agents. For example, the one or more mobilization agents in the compositions can be combined at each of these variable dosage amounts. In various embodiments, the composition includes 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of Hippophae rhamnoides or extracts thereof, Terminalia chebula or extracts thereof, Ribes spp. of extract thereof, and/or Panax notoginseng of extract thereof, analogs thereof, derivatives thereof, synthetic or pharmaceutical equivalents thereof, fractions thereof, and combinations of any of the foregoing items.

In various embodiments, the dosages can contain one or more mobilization agents for a total amount of 50-250, 250-500, 500-750, 750-1000, 1000-2000, 2000-3000, 3000 mg or more. For example, in various embodiments, the pharmaceutical composition includes 750 mg or less of Hippophae rhamnoides or extracts thereof, 1000 mg or less of Terminalia chebula or extracts thereof, 750 mg or less of Ribes spp. or extracts thereof and 500 mg or less of Panar notoginseng or extracts thereof. In various embodiments, the total dosage amount is administered daily for one or more days, or multiple times in a single day.

In some embodiments, the subject administered a mobilization agent is healthy. In other embodiments, the subject is suffering from a disease or physiological condition, such as immunosuppression, chronic illness, traumatic injury, degenerative disease, infection, or combinations thereof. In certain embodiments, the subject may suffer from a disease or condition of the skin, digestive system, nervous system, lymph system, cardiovascular system, endocrine system, or combinations thereof. In specific embodiments, the subject suffers from osteoporosis, Alzheimer's disease, cardiac infarction, Parkinson's disease, traumatic brain injury, multiple sclerosis, cirrhosis of the liver, any of the diseases and conditions described in the Examples below, or combinations thereof. Administration of a therapeutically effective amount of a mobilization agent may prevent, treat and/or lessen the severity of or otherwise provide a beneficial clinical benefit with respect to any of the aforementioned conditions, although the application of the inventive methods and use of the inventive mobilization agent is not limited to these uses. In various embodiments, the compositions and methods of the present disclosure find therapeutic utility in the treatment of, among other things, skeletal tissues such as bone, cartilage, tendon and ligament, as well as degenerative diseases, such as Parkinson's and diabetes.

The present invention further provides various compositions for administration to a subject. In one embodiment, the administration is topical, including ophthalmic, vaginal, rectal, intranasal, epidermal, and transdermal. In one embodiment, the administration is oral. In one embodiment, the composition for oral administration includes powders, granules, suspensions or solutions in water or non-aqueous media, capsule, sachets, tablets, lozenges, or effervescents. In another embodiment, the composition for oral administration further includes thickeners, flavoring agents, diluents, emulsifiers, dispersing aids or binding agents.

Described herein are mobilization agents and methods of using mobilization agents towards promoting stem cell trafficking. Described herein are releasing agents and methods of using releasing agents to promote egress of stem cells from a tissue of origin. Also described herein is a method of oral administration of mobilization agents which result in a significant release of stem cells into peripheral blood circulation. The present disclosure provides methods for the effective administration of stem cell mobilization agents, thereby achieving a safe, convenient and effective method to enhance stem cell-related maintenance and repair in the human body. Although the pathology of stem cells is of great importance and interest, and pertains to the subject matter disclosed herein, the underlying scope of this invention is that the release of stem cells from tissues, including the bone marrow, and the increase in the number of circulating stem cells is of significance in repairing injured tissue and maintaining the vitality and health of existing tissue. Thus, the importance of developing methods and compositions for achieving this end are among the foci and aims of the present invention.

Accordingly, the present invention provides methods for, among other things, enhancing natural tissue healing and renewal in the body by supporting the mobilization and trafficking of stem cells. Furthermore, the present invention provides novel methods for preventing, slowing or otherwise diminishing the development of health problems in a mammal by promoting mobilization and trafficking of stem cells in the mammal. The methods disclosed herein may further increase regeneration of existing tissue by supporting the release, circulation, homing and/or migration of stem cells into tissue, therefore supporting the process of tissue repair.

### V. Examples

### Example 1—Production and Preparation of Hippophae rhamnoides Extract and/or Ribes Spp. Fruit Extract

Extraction can be performed using Ultrasound-Assisted Extraction, which allows permeation of intracellular compounds and therefore liberation of polyphenols and other molecules. Since polyphenols and more specifically anthocyanins are vacuolar pigments, which accumulate in the plant cell central vacuole, cavitation and cell disruption caused by ultrasound waves enhances the mass transfer from the solid matrix to the solvent improving the extraction of anthocyanins.

In brief, dried fruit powder or dried leaves powder of Hippophae rhamnoides and/or dried fruit powder of Ribes spp. is mixed 1:50 with water maintained at a temperature ranging from 5° C. to 60° C. (35° C.), passed through high-sheer homogenization at 3,000 to 12,000 RPM (9,000 RPM), and then sonicated at 10 to 40 kHz (20 kHz) for 10 to 60 minutes. The solvent (water) is then filtered using conventional filtration methods and dried.

### Example 2—Prophetic Alternative Methods for Production and Preparation of Hippophae rhamnoides Extract and/or Ribes Spp. Fruit Extract

The following examples are prophetic and should be considered as such. The following prophetic examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Fresh Hippophae rhamnoides fruit or leaves (20 g), or fresh Ribes spp. fruit (20 g) were used for the preparation of extract. The edible part of the fruit was carefully removed and chopped into fine pieces. Likewise, fresh leaves can be chopped into fine pieces. 50 ml (80% v/v) acidic methanol (Methanol and 1 N HCl in 80:20) was added to the fruit or leave part and kept in a water bath (60 C) for 1 h. After cooling, the supernatant was kept under continuous shaking condition at room temperature for 15 h. The extract was filtered and a filtrate was centrifuged at 8000 rpm (10,000 g) for 10 min. The supernatant was stored at 4 C and used for quantification of total phenolics, flavonoids and antioxidant activity within two days of extraction.

Total phenolics were measured by transferring a small portion (0.25 ml) of the methanolic extract into a test tube containing 2.25 ml of distilled water, followed by the addition of 0.25 ml of Folin-Ciocalteu's reagent and allowed to stand for reaction for 5 min at 22±1 C. The mixture was neutralised by adding 2.50 ml of 7% (w/v) sodium carbonate and kept in the dark at 22±1 C for 90 min. The absorbance of the resulting colour solution was measured at 765 nm using a spectrophotometer (Hitachi U-2001, Tokyo, Japan). Quantification was based on a standard curve of Gallic acid prepared in 80% (v/v) methanol and the results were expressed in mg Gallic acid equivalents (GAE) per g of fresh weight of the fruits.

Total flavonoids were determined by the aluminium chloride colorimetric method whereby the extract (0.50 ml) was diluted with 1.50 ml of distilled water, followed by the addition of 0.50 ml of 10% (w/v) chloride, 0.10 ml of 1.0 M potassium acetate and 2.80 ml of distilled water. This mixture was incubated at 22±2 C for 30 min and its absorbance was measured at 415 nm using a spectrophotometer (Hitachi U-2001, Tokyo, Japan). Quantification carried out on the basis of the standard curve of quercetin prepared in 80% (v/v) methanol and the data were expressed in mg quercetin equivalents (QE) per g of fresh weight (fw) of the fruits.

For quantification of anthocyanins, 5 g of fruit or leaf sample was homogenized with 50 ml of 80% (v/v) acidified ethanol (95% ethanol: 1.5 N HCl). Samples were allowed to stand for overnight incubation at room temperature followed by filtering the extract and samples were stored at 4 C until analysis within a week. The total anthocyanins content were quantified by pH differential method. A UV-vis spectrophotometer (Hitachi U-2001, Tokyo, Japan) was used to measure the absorbance at 510 and 700 nm. Anthocyanin levels expressed as mg of cyanidin-3-glucoside equivalents per g of fresh weight (mg CGE/g fw), using the reported molar extinction coefficient of 26,900 Ml per cm and a molecular weight of 449.2 g/mol.

### Example 3—a Prophetic Method for the Production and Preparation of Terminalia chebula Extract

The effect of temperature and duration of extraction were first optimized using water as an extraction solvent. In a typical experiment, dried whole fruit and fruit pericarp portions of T. chebula (50 g) were pulverized and the resulting powder was extracted separately with distilled water (300 ml) for 16 hours at room temperature, kept without stirring.

Thus, the above extraction procedures yielded an enriched hydrolyzable tannoid blend. It is exemplary that other temperatures may be useful for all-aqueous extraction, such as, for example, 300° C., 40° C., 50° C., 60° C., 70° C., and 80° C. Useful extraction times may range from about 0 hours to about 24 hours. Other suitable extraction times may range from about 0 hours to about 6 hours, or from about 0 hours to about 4 hours, or from about 0 hours to about 3 hours, or from about 0 hours to about 2 hours. Additionally, the above extraction procedures yielded total extractive tannin or tannoid compositions, that is, T chebula/enriched tannoids of about 45% by weight, or greater, based on the total weight of the extract composition. In one embodiment, the total yield T. chebula/enriched tannoids is about 50% by weight, or greater.

Further details regarding this and other methods for the extraction of T. chebula can be found in U.S. patent application Ser. No. 13/859,633, which is incorporated by reference herein.

### Example 4—Prophetic Alternative Methods for the Production and Preparation of Terminalia chebula Extract

Tannin extracts were temperature-sensitive. According to requirements of production, temperature during extraction was set to 50° C., and water was considered as the extraction solvent. Four factors can affect extraction: (A) duration of extraction, (B) maceration time, (C) extract-solvent ratio, and (D) number of extraction. The study was conducted in accordance with the orthogonal test of four factors at three different levels. The fruit powder of Terminalia chebula (10 g in weight) was extracted with water (100 ml) at 50° C. Thereafter, the extracts were weighted. Content of tannin extracts was measured and optimal extraction and purification technology (OEPT) was determined. The extracts were added to ethanol of 95% concentration, and the concentration of extract solutions were diluted to 80%. The extract solution was deposited for 12 hours and centrifuged at 4000 rpm for 10 min. After filtration, the content of tannin extracts was analyzed by the casein method.

### Example 5—General Study Design for Hippophae rhamnoides and/or Terminalia chebula and/or Ribes Spp. and/or Panax notoginseng as Stem Cell Mobilization Agent

Five consumables were tested in human subjects: a proanthocyanidin extract from Hippophae rhamnoides, a tannin extract of Terminalia chebula, a polyphenol extract of Ribes spp., a saponin extract of Panax notoginseng and a placebo. Peripheral venous blood samples were obtained from healthy human volunteers between 20 and 45 years of age upon informed consent. Five hundred milligrams of Hippophae rhamnoides extract or 500 milligrams of a tannin extract of Terminalia chebula or 500 milligrams of a polyphenol extract of Ribes spp. or 250 milligrams of a saponins extract of Panar notoginseng or placebo was given to volunteers with 4-6 oz water. Appearance of the placebo was identical to that of the extracts and consisted of orange-dyed or brown-dyed, finely ground potato flakes encapsulated in vegetable capsules. The number of stem cells in circulation was quantified at 0, 60, 120, and 180 minutes after consumption of the consumables. For each participant, the response to the consumables is compared to the placebo.

### Example 6—In Vivo Study Design

The following exclusion criteria were used: under 20 or over 70 years of age, pregnancy, severe asthma and allergies requiring daily medication, any known chronic illness or previous/current venereal disease, frequent recreational drug use, and impaired digestive function (including previous major gastrointestinal surgery). Four volunteers were scheduled on two study days, separated by a wash-out period of at least 1 week. Testing was always performed at the same time of the day (8-11 a.m.) to minimize the effect of circadian fluctuations. Due to the interference from stress with the release vs. homing of other types of lymphocytes, effort was taken to minimize any physical and mental stress during testing. In addition, on each study day, volunteers were instructed to complete a questionnaire aimed at determining any exceptional stress related circumstances that might affect the person on that particular study day. Predetermined criteria for exclusion from final analysis included significant lack of sleep and severe anxiety. After completing the questionnaire, volunteers were instructed to remain quiescent for 4 h, comfortably seated in a chair. After the first hour, the baseline blood sample was drawn. Immediately after drawing the baseline sample, a consumable was provided. Blood samples were later drawn 60 and 120 min after ingestion of the consumable. At each time point, 5 ml of blood was drawn into heparin, and 2 ml blood was drawn into EDTA. The blood vials were placed on a rocking plate until use.

### Example 7—Measurement of Stem Cell Populations Using FACS Sorting

The blood drawn into EDTA was used for obtaining a complete blood count (CBC) with differential, using a Coulter counter (Micro Diff II, Beckman Coulter). All CBCs were performed within an hour of drawing the sample. All CBCs were performed in triplicate. The heparinized blood was used for purification of the PBMC fraction by gradient centrifugation and processed for immunostaining and flow cytometry. The stem cell markers CD31-FITC, CD34-PerCP and CD309-PE were used for two color immunofluorescence. Staining of all samples with CD34-FITC/CD309(KDR) was performed in triplicate. IgG1-FITC and IgG1-PE isotype controls were used in parallel samples. Separate, positive control samples for each donor was done using CD45-FITC. Stained PBMC were fixed in 1% formalin and acquired by flow cytometry immediately. Files of 200,000 events were collected on each triplicate sample. The percent CD34+CD309−, CD34+CD309+, and the CD45dimCD34+ subsets were analyzed separately and were analyzed again after multiplying with the lymphocyte cell counts, as obtained from the average of the triplicate lymphocyte counts obtained by the CBC differential count.

### Example 8—Increase in CD34⁺ HSCs Circulating in Peripheral Blood Following Oral Administration of Hippophae rhamnoides Extract

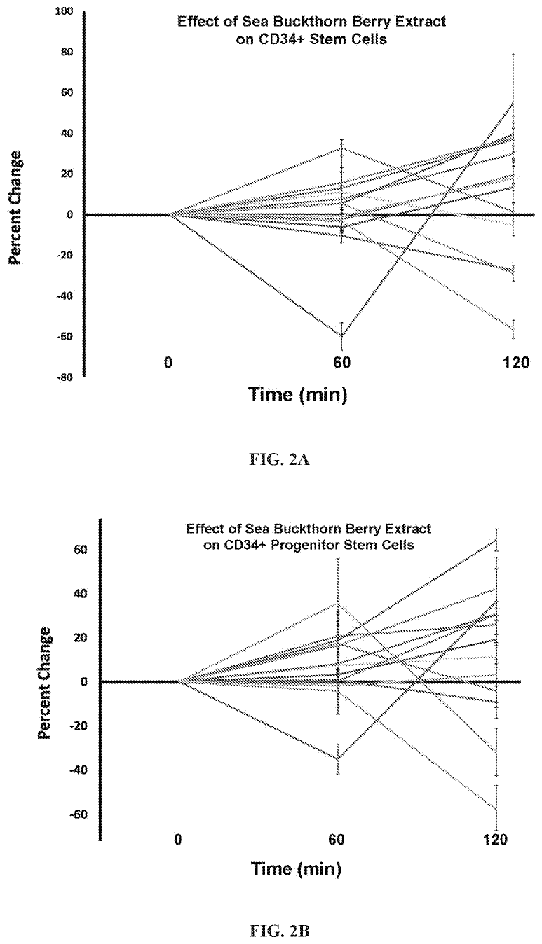

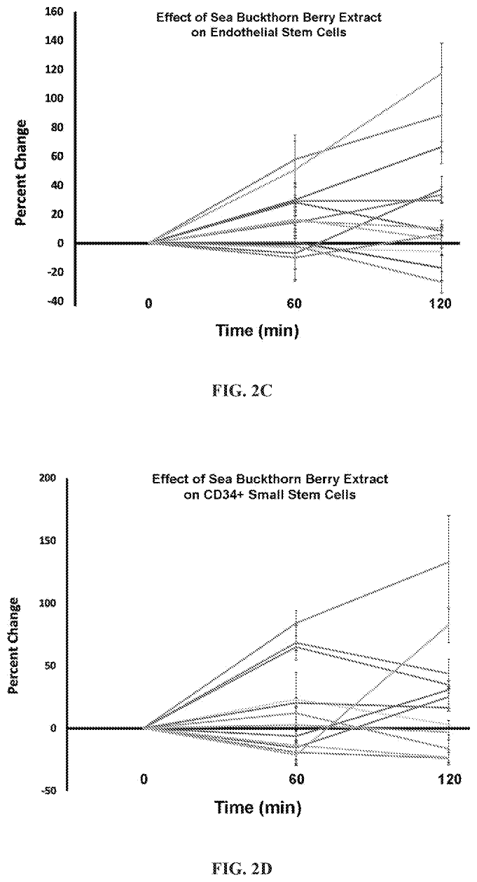

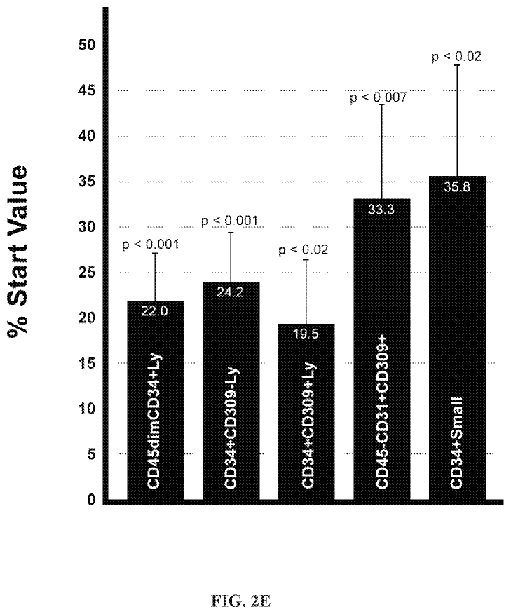

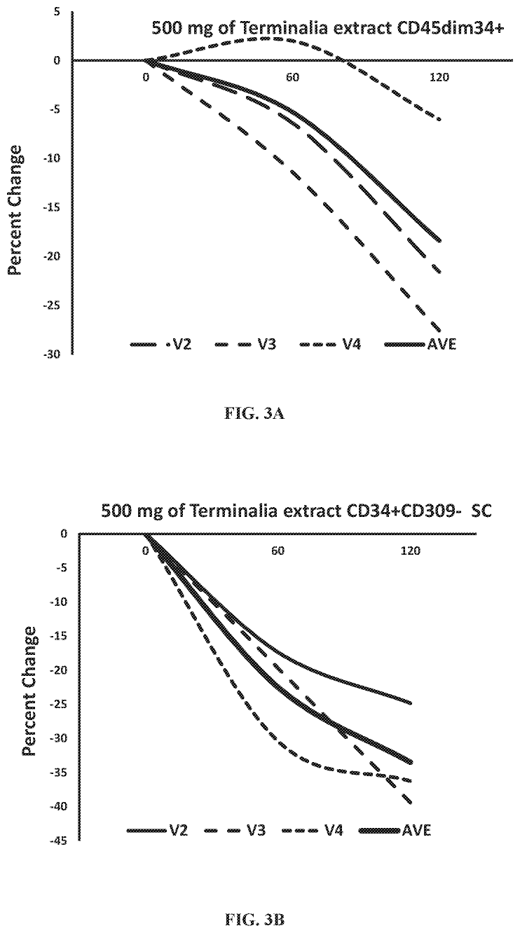

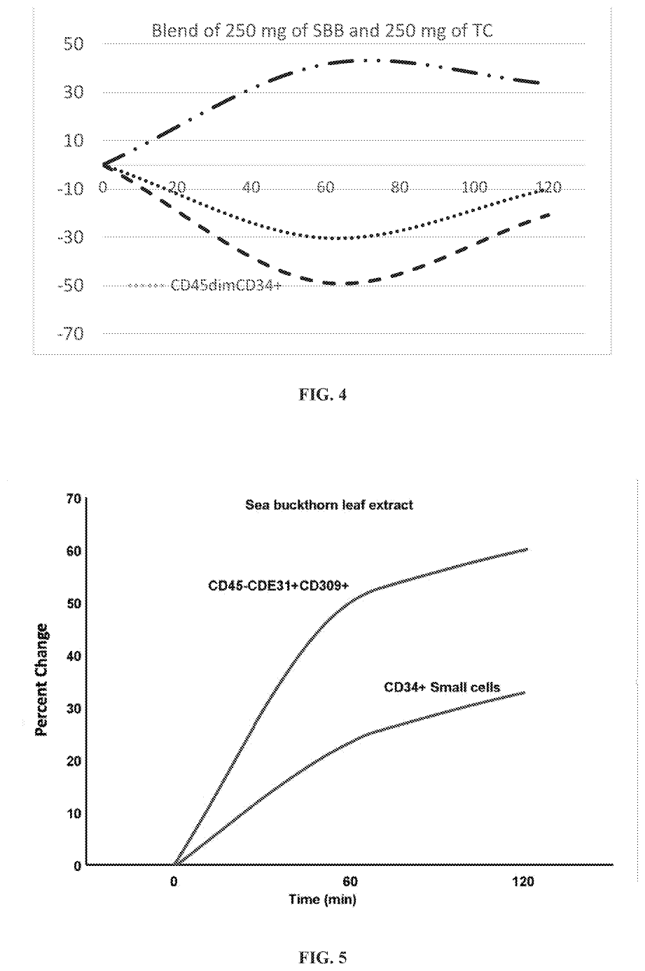

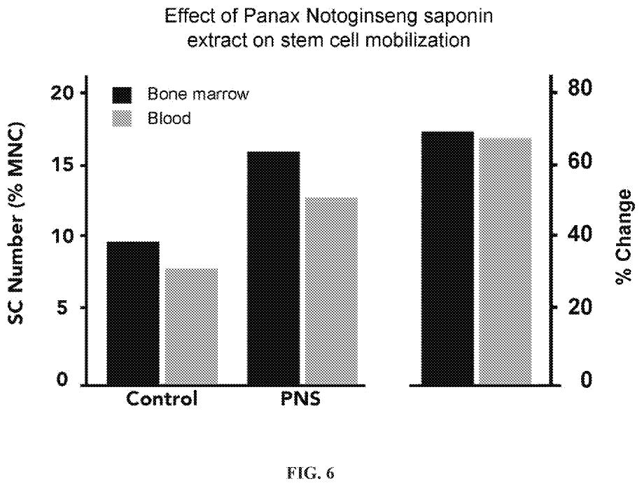

Oral administration of Hippophae rhamnoides fruit extract was tested for its potential to effectuate stem cell mobilization in the peripheral bloodstream of human subjects. Hippophae rhamnoides extract resulted in a significant elevation in the number of small CD34⁺ stem cells of 30.6% and 53.5% occurring after 60 and 120 minutes, thereby demonstrating efficacy as a releasing agent (FIG. 2D). Hippophae rhamnoides fruit extract also resulted in a significant elevation in the number of CD³¹⁺CD³⁰⁹⁺ stem cells of 16.8% and 26.8% occurring after 60 and 120 minutes (FIG. 2C). Hippophae rhamnoides fruit extract resulted in a significant elevation in the number of CD³⁴⁺CD³⁰⁹⁻ stem cells of 34.5% occurring after 120 minutes (FIG. 2B). Hippophae rhamnoides fruit extract resulted in a significant elevation in the number of CD^{45dim}CD³⁴⁺ stem cells of 16.8% occurring after 120 minutes (FIG. 2A). Altogether this data demonstrates efficacy of Hippophae rhamnoides extract as a releasing agent.

Oral administration of Hippophae rhamnoides leaf extract was tested for its potential to effectuate stem cell mobilization in the peripheral bloodstream of human subjects. Hippophae rhamnoides leaf resulted in a significant elevation in the number of CD³¹⁺CD³⁰⁹⁺ stem cells of 52.3% and 58.6% occurring after 60 and 120 minutes, thereby demonstrating efficacy as a releasing agent (FIG. 5). Hippophae rhamnoides leaf resulted in a significant elevation in the number small CD34⁺ stem cells of 32.2% and 44.6% occurring after 60 and 120 minutes, thereby demonstrating efficacy as a releasing agent (FIG. 5).

### Example 9—Increase in CD34⁺ Stem Cells Circulating in Peripheral Blood Following Oral Administration of Terminalia chebula Extract

Oral administration of Terminalia chebula extract was tested for its potential to effectuate stem cell mobilization in the peripheral bloodstream of human subjects. Terminalia chebula extract resulted in a significant elevation in the number of small CD³⁴⁺ stem cells of 38.5% and 40.7% occurring after 60 and 120 minutes, thereby demonstrating efficacy as a releasing agent. Terminalia chebula extract also resulted in a significant elevation in the number of CD³¹⁺CD³⁰⁹⁺ stem cells of 10.1% and 31.2% occurring after 60 and 120 minutes. Altogether this data demonstrates efficacy of Terminalia chebula extract as a releasing agent.

### Example 10—Increase in CD34⁺ Stem Cells Circulating in Peripheral Blood Following Oral Administration of Panax notoginseng Extract

Oral administration of Panax notoginseng extract was tested for its potential to effectuate stem cell mobilization in the peripheral bloodstream of human subjects. Panax notoginseng extract resulted in a significant elevation in the number of CD³⁴⁺ stem cells of 50% occurring after 120 minutes, thereby demonstrating efficacy as a releasing agent.

### Example 10—Stem Cells from Bone Marrow Populate Multiple Distant Tissues

A murine model is chosen to evaluate how Hippophae rhamnoides fruit and/or leaf extract, Terminalia chebula extract, Ribes spp. extract, Panax notoginseng extract or a mixture of these extracts can stimulate stem cell release, and therefore support the repair of distant tissues of the body.

Male mice are selected as bone marrow donor animals, while all recipient mice are females. Female recipients are sub-lethally irradiated prior to injection of GFP⁺ male bone marrow cells into their tail veins. Two groups of mice are evaluated. The first group of 20 animals are sub-lethally irradiated, injected with bone marrow, and put on normal feed. The second group of 20 animals are also sub-lethally irradiated, receive male bone marrow, and are fed a diet of normal feed plus a mixture of Hippophae rhamnoides extract, Terminalia chebula extract, Ribes spp. extracts and Panar notoginseng extract. Incorporation of GFP⁺ cells is examined in the brain, heart muscle, muscles, liver, pancreas, sections of small intestine, and lung tissue.

These data document the extent to which a diet containing a mixture of Hippophae rhamnoides extract, Terminalia chebula extract, Ribes spp. extracts and Panax notoginseng extracts promotes the repair of various tissues.

### Example 11—Increased Stem Cell Repopulation of Traumatized Tissue

A murine model is chosen to evaluate how a mixture of Hippophae rhamnoides extract, Terminalia chebula extract, Ribes spp. extracts and Panax notoginseng extracts can stimulate stem cell release from the bone marrow and therefore stimulate the repair of distant tissues of the body.

Male mice are selected as bone marrow donor animals, while all recipient mice are females. Female recipients are sub-lethally irradiated prior to injection of GFP⁺ male bone marrow cells into their tail veins. Two groups of mice are evaluated. The first group of 20 animals are sub-lethally irradiated, injected with bone marrow, and put on normal feed. The second group of 20 animals are also sub-lethally irradiated, receive male bone marrow, and are fed a diet of normal feed plus a mixture of Hippophae rhamnoides extract, Terminalia chebula extracts, Ribes spp. extracts and Panax notoginseng extracts.

After bone marrow transplant and a few days prior to the initiation of the feeding trial, animals are subjected to an injury such as injection of cardiotoxin in the tibialis muscle, triggering of heart attack by ligation of coronary artery, punch of the skin, laser-induced stroke, or other injuries. The recovery of mice in both groups is monitored during 6 weeks using whole body fluorescence imaging. After 6 weeks, the animals are sacrificed and the injured tissue is analyzed to assess the extent of tissue repair. Incorporation of GFP⁺ cells will also be examined in the brain, heart muscle, muscles, liver, pancreas, sections of small intestine, and lung tissue.

These data document the extent to which a diet containing a mixture of Hippophae rhamnoides extract, Terminalia chebula extracts, Ribes spp. extracts and Panax notoginseng extracts promotes the release of bone marrow stem cells and enhance the process of tissue repair and healing.

### Example 12—Example Components of a Nutraceutical Composition

Base in part of the studies detailed herein, an example nutraceutical composition can be formulated having components as set forth below.

TABLE 1

| Ingredient | mg/dose | range (mg) |
|---|---|---|
| Hippophae rhamnoides (such as having at | 500 | 150-1000 |
| least 30% polyphenols & proanthocyanidins) | | |
| AFA extract | 300 | 250-1000 |
| Aloe extract (e.g., high poly acetyl mannans | 100 | 100-400 |
| concentration) | | |
| Fucus vesiculosus extract (such as having | 250 | 150-1000 |
| at least 20% phlorotannins) | | |
| Panax notoginseng extract (such as having | 125 | 100-500 |
| at least 20% saponins) | | |
| Beta-glucan (85%) | 100 | 50-500 |

### Example 13—Clinical Trial Design and Results

Given the wide range of benefits associated with the use of Hippophae rhamnoides, in this study we investigated the effect of the consumption of a proanthocyanidin-rich extract of Hippophae rhamnoides (SBB-PE) on stem cell mobilization. The clinical study followed a randomized, double-blinded, placebo-controlled crossover design. Twelve people were screened and enrolled upon written informed consent, as approved by the Sky Lakes Medical Center Institutional Review Board (FWA 2603). The trial is registered at clinicaltrials.gov (ClinicalTrials.gov Identifier: NCT03388073).

A. Study Design, Consumables, and Analytical Methods

The study population included eight females and four males with an average age of 53.4±12.3 years and a body mass index (BMI) between 21.0 and 34.6 kg/m2 (Table 2), with no known chronic illness, frequent recreational drug use, impaired digestive function (including previous major gastrointestinal surgery) or known allergies to berry products.

TABLE 2
Demographics of the Study Population

| Participant | | Age | BMI |
|---|---|---|---|
| ID | Gender | (years) | (kg/m2) |
| P01 | F | 56 | 32.5 |
| P02 | F | 62 | 23.6 |
| P03 | F | 62 | 30.8 |
| P04 | F | 32 | 23.5 |
| P05 | M | 28 | 22.1 |
| P06 | M | 59 | 29.8 |
| P07 | M | 49 | 31.7 |
| P08 | F | 61 | 24.5 |
| P09 | M | 70 | 21.0 |
| P10 | F | 56 | 22.0 |
| P11 | F | 55 | 34.6 |
| P12 | F | 51 | 23.3 |
| Average | | 53.4 | 26.6 |
| Standard deviation | | 12.3 | 4.9 |
| Range | | 28-70 | 21.0-34.6 |

The study participants were scheduled for two clinic visits at least 1 week apart. Testing was always performed at the same time of the day for each person, the same day of the week, and always during the morning hours of 7-11 A.M. to minimize the effect of circadian fluctuations. Because of the interference from exercise (Yang and Kallio, 2001) and stress (Bal et al., 2011, Tulsawani et al., 2013, Dhyani et al., 2007, and Khan et al., 2010) with the release versus homing of lymphocytes, the study environment was tailored to avoid physical and mental stress prior to and during testing. On each study day, study participants completed a questionnaire to help monitor any exceptional stress-related circumstances that might be affecting the person on that day. Predetermined criteria for exclusion from data analysis included sleep deprivation and acute anxiety. After completing the questionnaire, volunteers were instructed to remain calm and inactive for 3 hours, comfortably seated in a chair. After the first hour, the baseline blood sample was drawn. Immediately after the baseline sample was drawn, an encapsulated test product was provided with water and consumed in the presence of clinic staff. Blood samples were drawn at 1 and 2 hours after ingestion of the test product or placebo. At each blood draw, 6 mL of blood was drawn into sodium heparin vacutainer tubes for subsequent immunostaining. The heparin vials were placed on a rocker until staining, which was initiated within the hour of each blood draw.

A proanthocyanidin-rich extract of Seabuckthorn berries (Puredia, Irvine, Calif., USA) was encapsulated at NIS Labs with 500 mg/dose. Placebo capsules were prepared using rice flour. Study participants and clinic staff were blinded to the consumables. Randomization was performed following the Latin Square design, such that half the study participants received the active test product at the first visit and placebo at the second visit, and the other half of the study participants received products in the reverse sequence.

For each blood draw, triplicate samples of 100 μL of heparinized whole blood were stained using the following 4-color immunostaining panel: CD31-FITC, CD34-PerCP, CD45-PO, and CD133-PE. For eight of the study participants, a fifth color was added: CD90-v421. Staining was performed as recommended by Life Technologies for whole blood staining followed by a ‘no-wash’ procedure involving Cal-Lyse® fixation of white blood cells and lysing of red blood cells. In brief, samples were stained in the dark at room temperature for 15 minutes followed by the addition of 100 μL of Cal-Lyse® Lysing solution and fixation for 10 minutes at room temperature. Red blood cells were then lysed by the addition of 1 mL of deionized water and further 10 minutes incubation in the dark at room temperature. Samples were stored at 4° C. in the dark and acquired by flow cytometry within 24 hours using an acoustic-focusing Attune™ flow cytometer (Life Technologies). Files of 300,000-600,000 events were collected for each triplicate sample. Data on stem cell numbers were analyzed by the Attune software that provides results as cell per microliter of sample and compensated for the dilution factor that was part of the immunostaining protocol, so the results were converted to stem cell numbers per microliter whole blood.

Average and standard deviation for each data set were calculated using Microsoft Excel. The statistical significance of post-consumption changes from baseline to later assessments was evaluated by between-treatment analysis using within-subject analysis and the two-tailed paired t-test. Statistical significance was indicated by P<0.05, and a high level of significance was indicated by P<0.01.

B. Results

The demographic characteristics for the study participants are shown in Table 2. All twelve study participants completed the study participation with full compliance, including adhering to similar routines and food for 12 hours prior to arrival on both clinic days, remaining calm and unstressed during the 3-hour clinic visits, consuming test products with water as instructed, and allowing the three blood draws at each visit.

Each blood sample was used to perform immunostaining and flow cytometry to evaluate the post-consumption changes to the numbers of three different subtypes of circulating stem cells. For eight of the study participants, post-consumption changes to a subset of mesenchymal stem cells, identified by the absence of CD45 and the expression of CD90 on cells within the lymphocyte gate as well as cells within the granulocyte gate, was also evaluated.

Given inter-individual variations in the time course of the mobilizing response following the consumption of SBB-PE, some individuals show a greater response at 60 minutes while others show a greater response at 120 minutes. Therefore, simply averaging the responses at the respective time points may underestimate the actual response and provide for greater standard deviations. For this reason, an additional analysis was done using only one data point, by selecting the greatest response at either 60 or 120 minutes.

CD34 is a transmembrane protein that is expressed almost exclusively on certain types of stem cells. Lymphocytoid CD34+ stem cells are also showing a low level of expression of the protein tyrosine phosphatase receptor type C enzyme, CD45 (CD45^{dim}). This is in contrast to mature hematopoietic cells that show a high level of cell surface CD45 expression, and to other types of stem cells that are completely negative for CD45 (CD45−). The changes in CD45^{dim} CD34+ stem cell numbers were analyzed to see whether the effects of consuming SBB-PE altered the levels of CD34+ stem cells in the circulation.

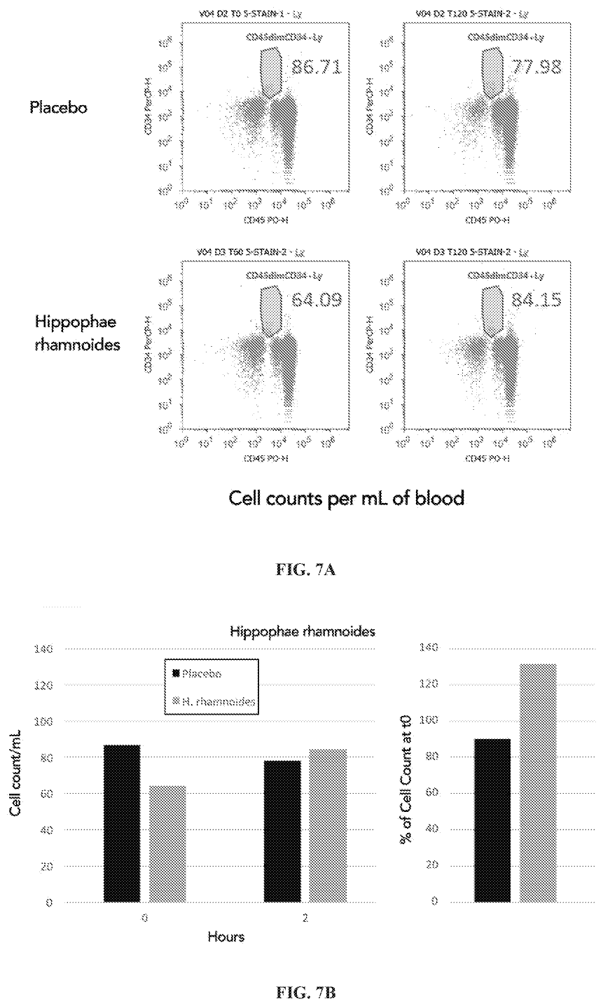

The CD45^{dim} CD34+ stem cell population is further divided into two subtypes, based on whether the cells express the CD309 antigen, a transmembrane tyrosine kinase also known as vascular endothelial growth factor receptor-2 (VEGFR-2) and kinase insert domain-containing receptor (KDR). The presence of CD309 on stem cells has been associated with a more pluripotent (i.e. undifferentiated) phenotype. Furthermore, expression of KDR on CD34+ cells in the circulation has been implicated with vascular maintenance and repair. The absence of the KDR (kinase insert domain-containing receptor, CD309) receptor on CD34+ stem cells has been associated with a progenitor phenotype. Consumption of SBB-PE triggered a selective mobilization of CD45dim CD34+CD309− cells, in contrast to no changes to CD45dim CD34+CD309+ pluripotential stem cells (see FIG. 7). When considering the greatest response at either 60 or 120 minutes, the number of circulating CD45dim CD34+CD309− cells and CD45dim CD34+CD309+ cells increased by an average 24.2±5.3% and 19.5±7.0%, respectively.

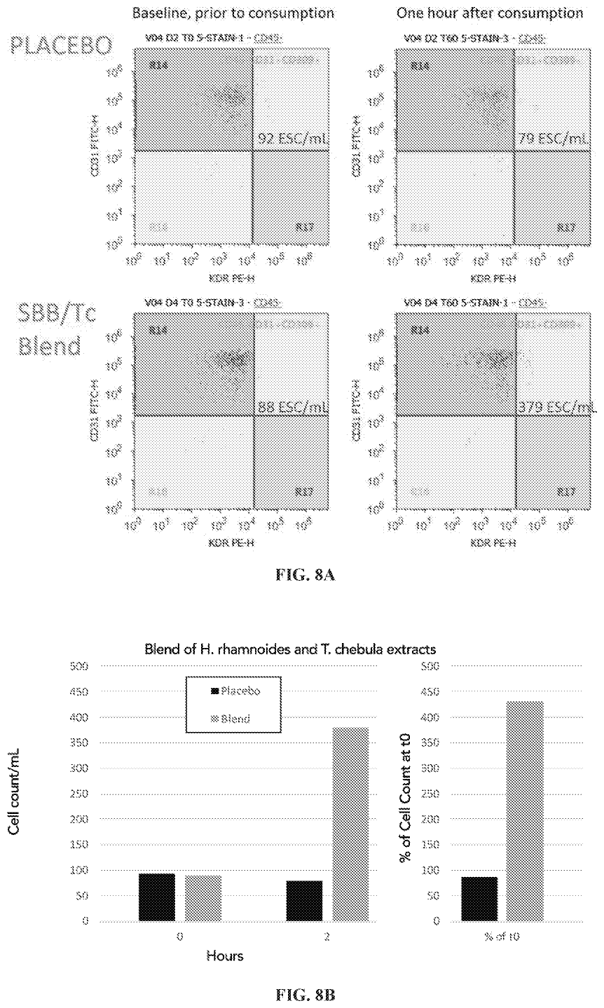

The immunophenotyping also included the assessment of circulating endothelial stem cells with the phenotype of CD45−CD31+CD309+. Endothelial stem cells have been shown to be rapidly mobilized following acute myocardial infarction, and are being actively researched for use in regenerative medicine. Consumption of SBB-PE triggered mobilization of CD45−CD31+CD309+ endothelial stem cells, in contrast to placebo where a mild reduction was seen, likely part of the normal circadian rhythm for this cell type. The difference in the numbers of circulating CD45−CD31+CD309+ endothelial stem cells after consumption of SBB-PE versus placebo was statistically significant at 1 hour (p<0.04) and after 2 hours (p<0.05) (see FIG. 8). When considering the greatest response at either 60 or 120 minutes, the number of circulating CD45−CD31+CD309+ cells increased by an average 33.4±10.2%.

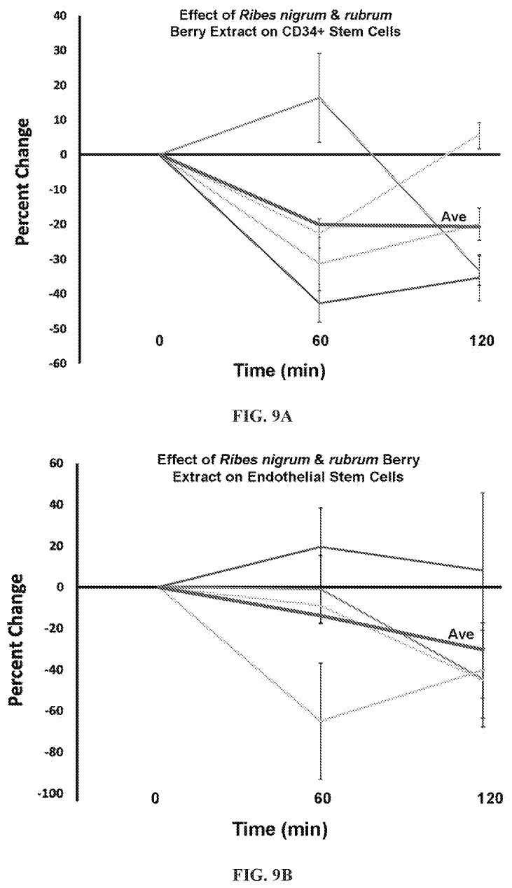

For eight of the twelve study participants, the changes in numbers of stem cells that expressed the mesenchymal stem cell marker CD90 was evaluated. Two separate subsets were identified, based on side scatter properties and absence versus presence of CD45 versus CD34. The low side scatter lymphocytoid stem cells that expressed the mesenchymal stem cell marker CD90 were negative for CD45. Post-consumption changes showed an increase in CD45−CD90+ stem cells at 1 hour and 2 hours after consuming SBB, in contrast to only minor changes after consuming placebo. The increase at 2 hours, compared to baseline, reached a borderline statistical trend (p<0.11). Among the cells with higher side scatter properties (granulocytes), a distinct subset was seen, co-expressing CD34 and CD90. In contrast to the lymphocytoid mesenchymal stem cells, a mild and non-significant increase was seen over the course of the 2-hour post-consumption evaluation for both SBB and placebo, suggesting that this slight increase is simply a normal part of the circadian changes, and not associated with SBB consumption (see FIGS. 9A & 9B). When considering the greatest response at either 60 or 120 minutes, the number of circulating CD45−CD90+ stem cells and CD34+CD90+ cells increased by an average 20.8±5.7% and 3.5±2.3%, respectively.

### Example 12—Antioxidant Protection and Immune Modulation

The antioxidant immune-modulating properties of a polyphenol extract of a blend of Ribes nigrum and Ribes rubrum berry were tested.

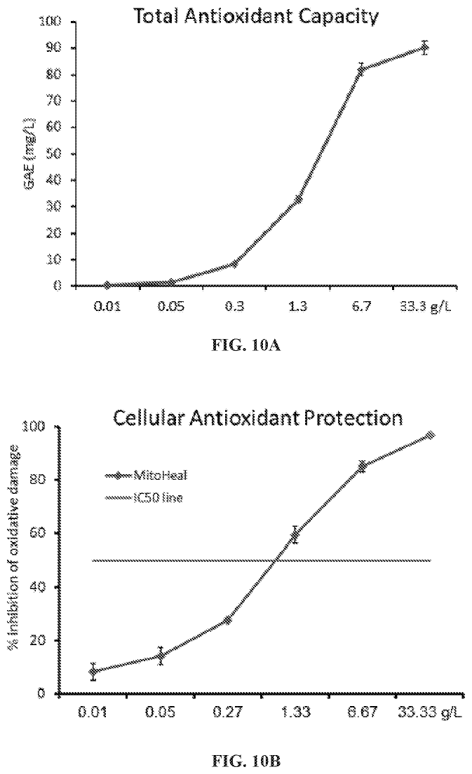

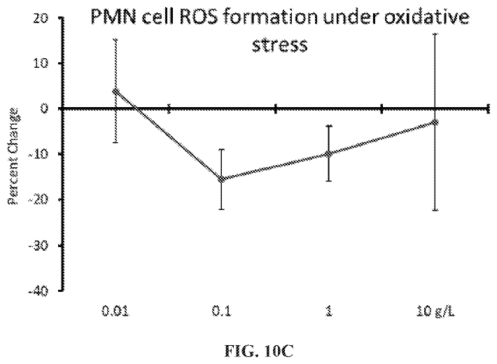

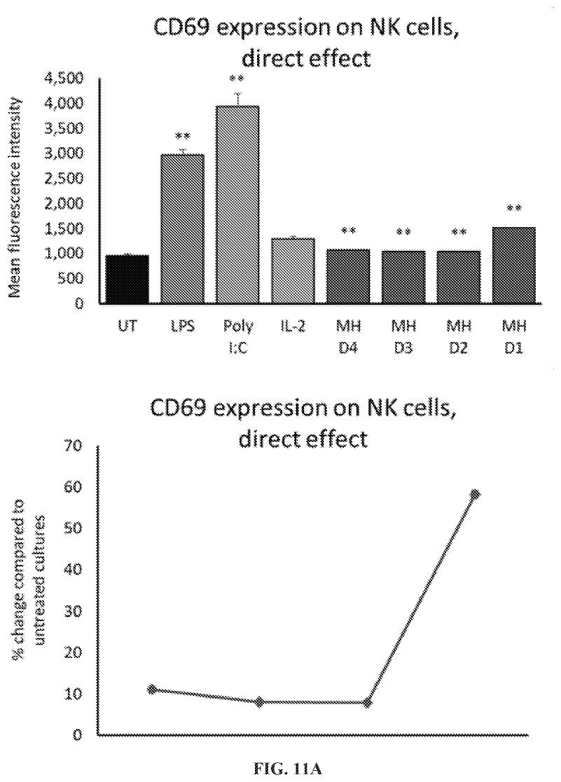

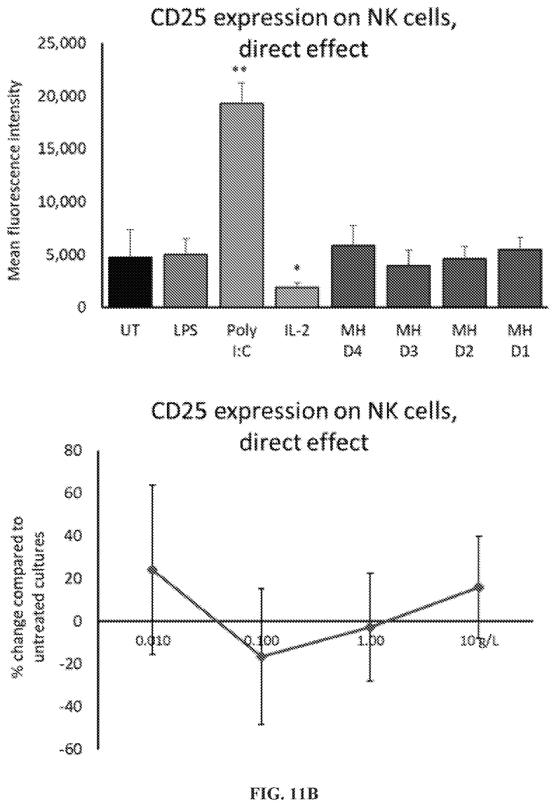

The Folin-Ciocalteu assay (also known as the total phenolics assay) was used to measure antioxidants (FIG. 10). The assay was performed by adding the Folin-Ciocalteu's phenol reagent to serial dilutions of extract, thoroughly mixing, and incubating for 5 minutes. Sodium carbonate is added, starting a chemical reaction producing a color. The reaction is allowed to continue for 30 minutes at 37° C. Optical absorbance is measured at 765 nm in a colorimetric plate reader. Gallic acid is used as a reference standard, and the data reported in Gallic Acid Equivalents per gram product. The extract was shown to have significant antioxidant capacity (FIG. 10A).

A CAP-e bioassay was designed specifically to work with natural products and ingredients. Red blood cells were used to assess antioxidants from complex natural products in a cell-based system. Freshly purified human RBC were washed repeatedly in physiological saline, and then exposed to the test products. During the incubation with a test product, any antioxidant compounds able to cross the cell membrane can enter the interior of the RBC. Then the RBC were washed to remove compounds that were not absorbed by the cells, and loaded with the DCF-DA dye, which turns fluorescent upon exposure to reactive oxygen species. Oxidation was triggered by addition of the peroxyl free radical generator AAPH. The fluorescence intensity was evaluated. The low fluorescence intensity of untreated control cells served as a baseline, and RBC treated with AAPH alone served as a positive control for maximum oxidative damage. It was observed that the extract had antioxidants available to penetrate the cells the protect from oxidative damage (FIG. 10B).

Human polymorphonuclear (PMN) cells were used for testing effects of a product on ROS formation. Freshly purified human PMN were exposed to the test products. During the incubation with a test product, any antioxidant compounds able to cross the cell membrane can enter the interior of the PMN cells, and compounds that trigger a signaling event can do so. Then the cells were washed, loaded with the DCF-DA dye, which turns fluorescent upon exposure to ROS. Formation of ROS was triggered by addition of H2O2. The fluorescence intensity of the PMN cells was evaluated by flow cytometry. The low fluorescence intensity of untreated control cells served as a baseline and PMN cells treated with H₂O₂ alone served as a positive control. As the fluorescence intensity of PMN cells exposed to an extract, and subsequently exposed to H2O2, was reduced compared to H₂O₂ alone, this indicated that the extract had anti-inflammatory effects (FIG. 10C).

Next, the immune-modulating effects of the extract were tested. Human peripheral blood mononuclear cell (PBMC) cultures were used for this testing. A set of cultures were left untreated as negative control cultures for immune activation. Triplicate sets of cultures were treated with serial dilutions of the test product. The inflammatory bacterial lipopolysaccharide LPS from E. coli was used as a positive control for activation. The cultures were incubated for 24 hours, after which the cells and the culture supernatants were harvested and used to monitor the reactions in each culture. The cells were used for immunostaining and flow cytometry, specifically to evaluate the induction of two activation markers, CD69 and CD25, on NK cells, NKT cells, T lymphocytes, and monocytes/macrophages.

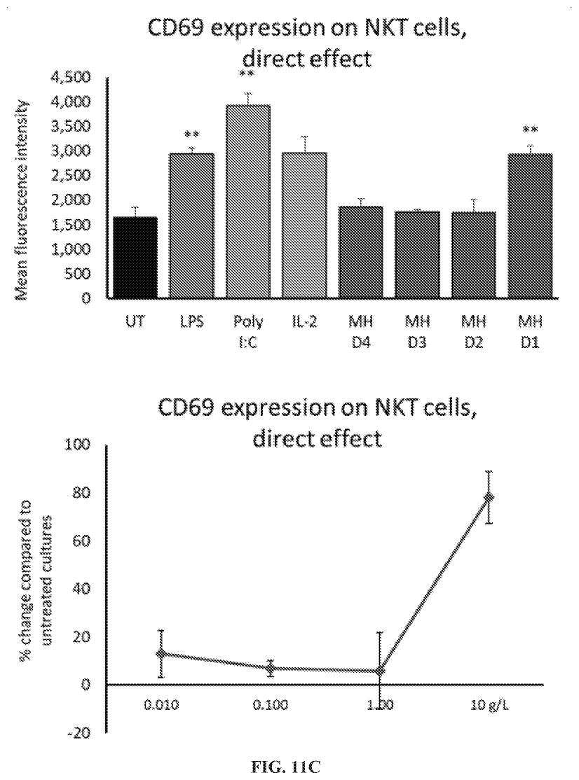

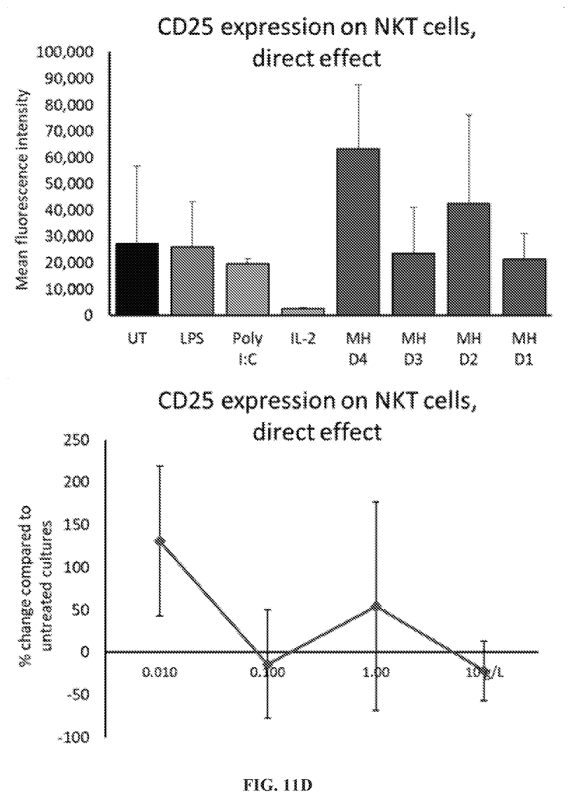

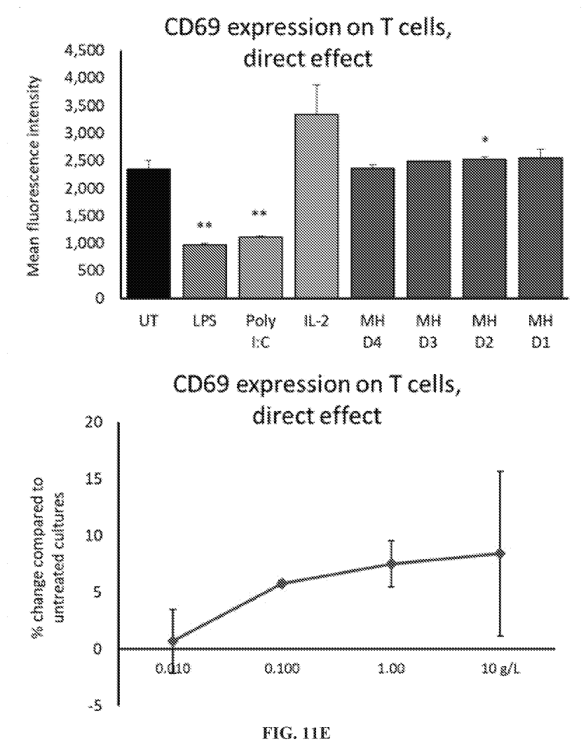

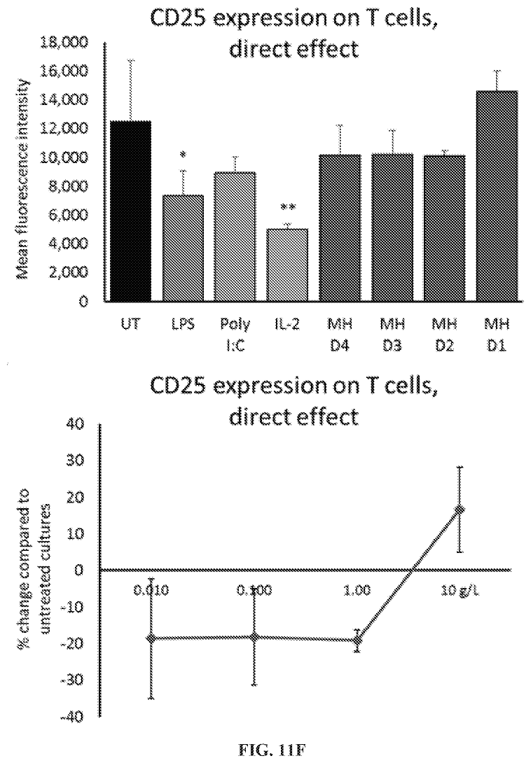

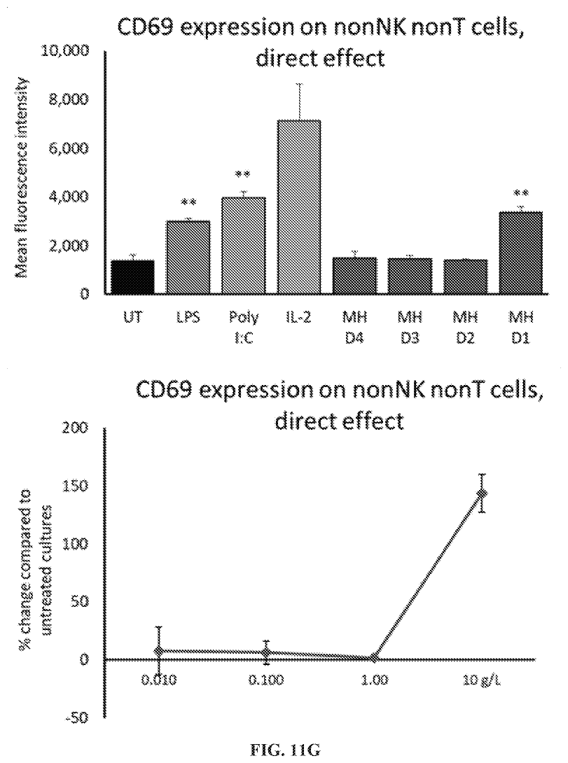

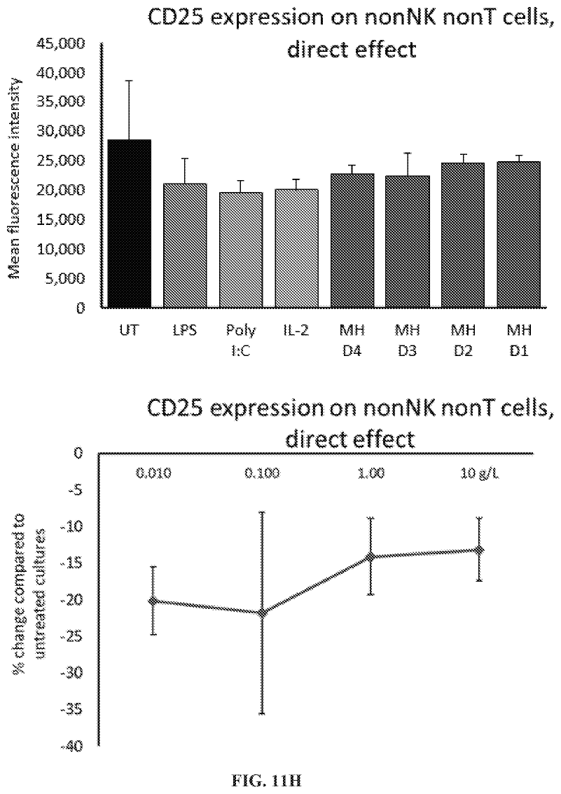

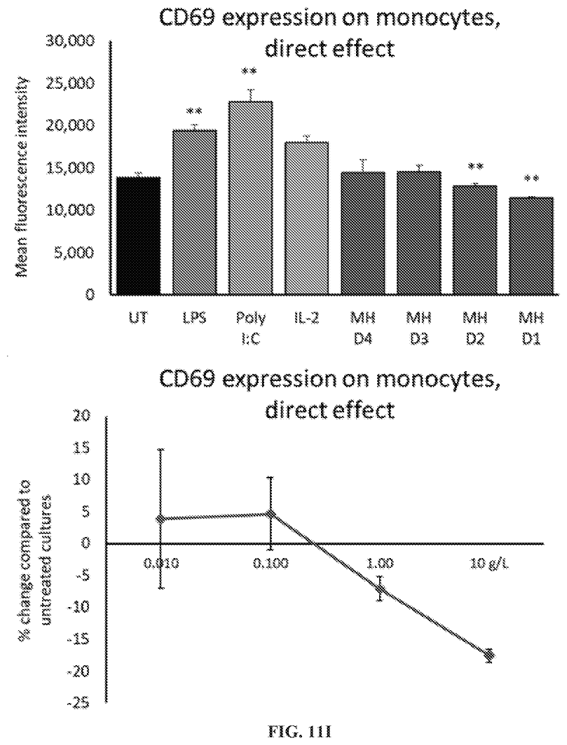

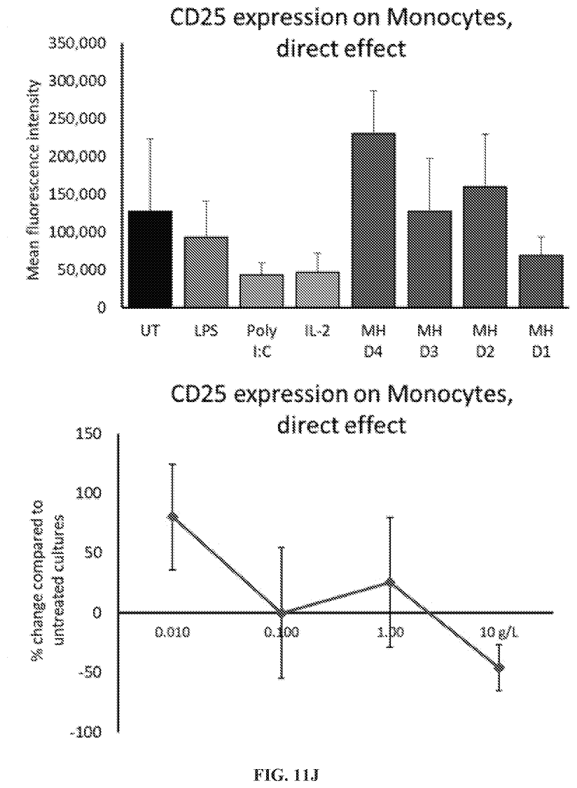

As expected, the three controls IL-2, LPS, and Poly I:C triggered an increase in CD69 expression on NK cells. At the highest dose, the extract showed an increase in CD69 on NK cells (FIG. 11A) and NKT cells (FIG. 11C). There was no significant change to CD25 expression on NK cells (FIG. 11B) or NKT cells (FIG. 11D). The extract also increased CD69 on T cells (FIG. 11E) not no significant changes to CD25 on T cells (FIG. 11F). In addition, the extract triggered a very significant increase in CD69 expression on non-NK, non-T cells (e.g., B lymphocytes and dendritic cells) (FIG. 11G) and a mild decrease in CD25 expression (FIG. 11H). The extract also triggered a decrease in CD69 expression on monocytes (FIG. 11I) and a mild increase in CD25 (FIG. 11H).

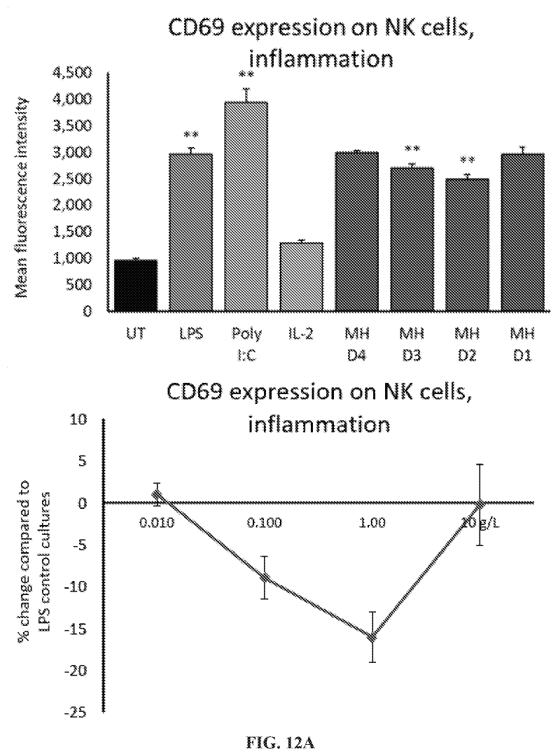

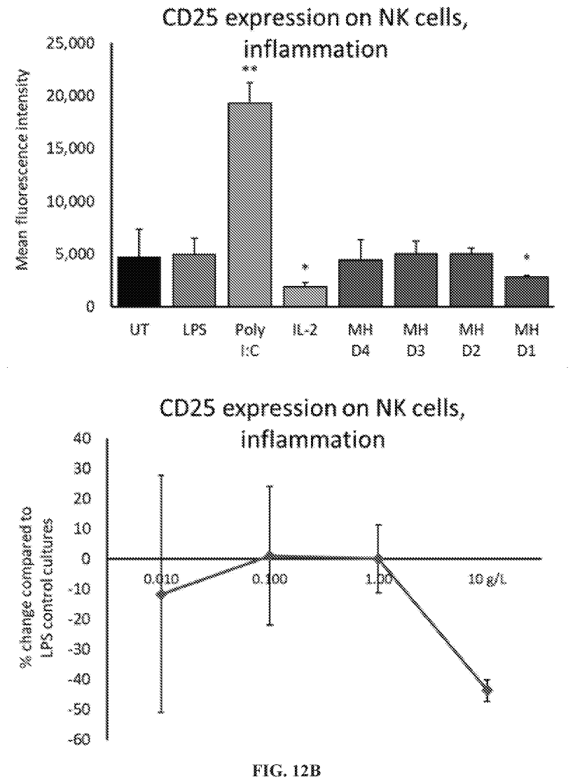

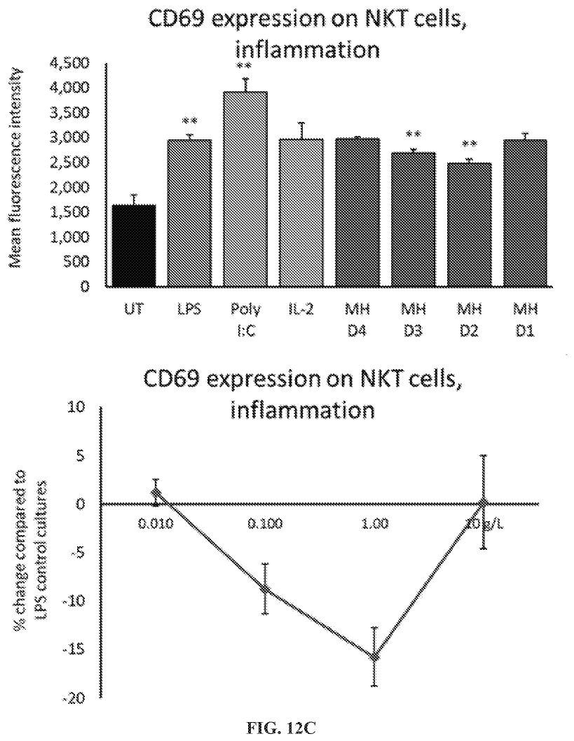

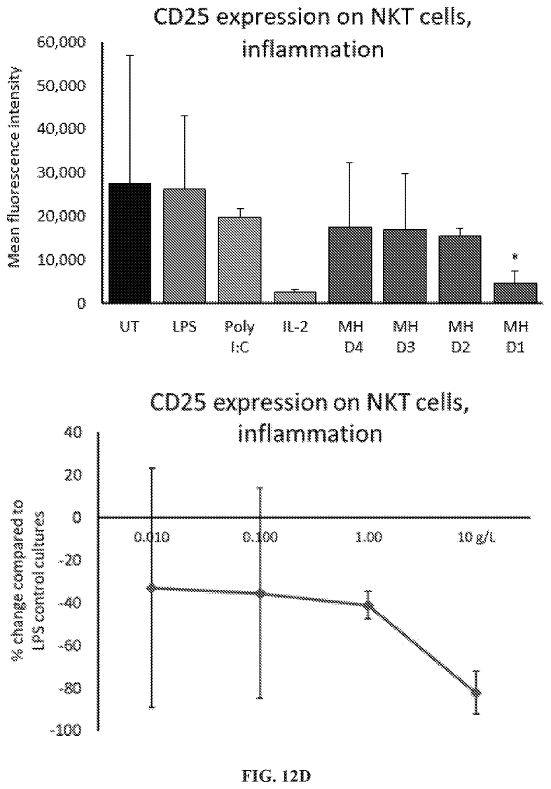

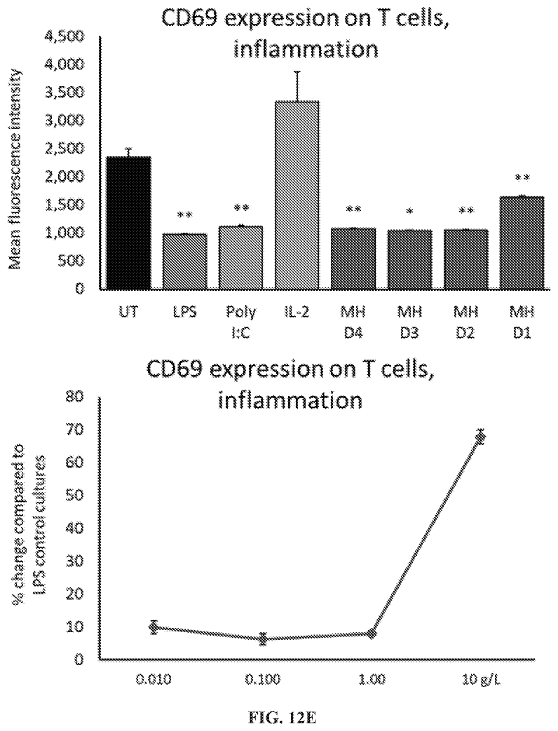

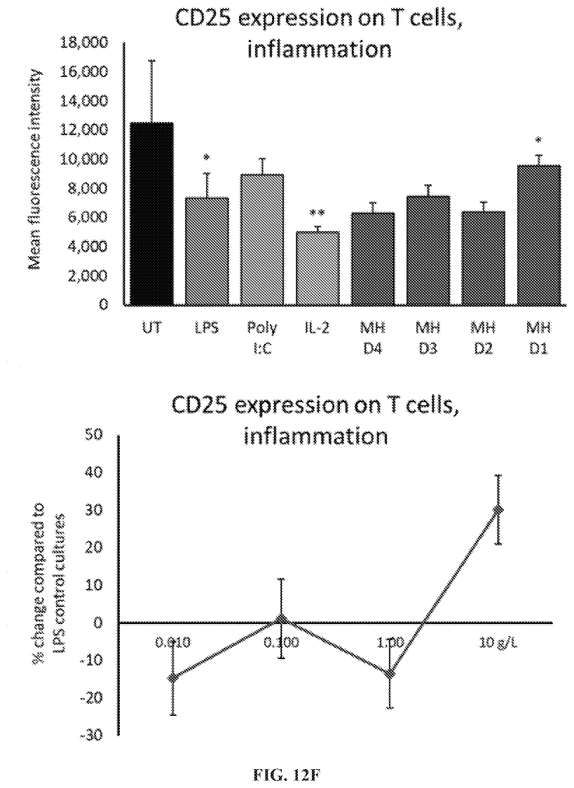

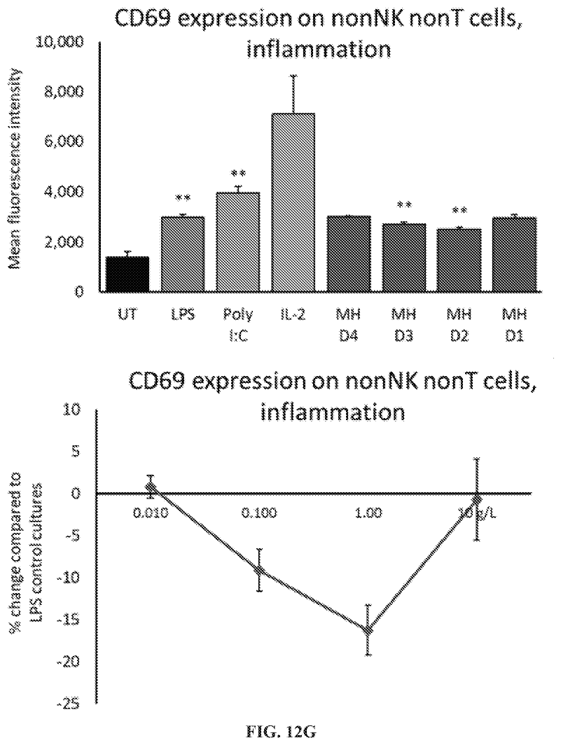

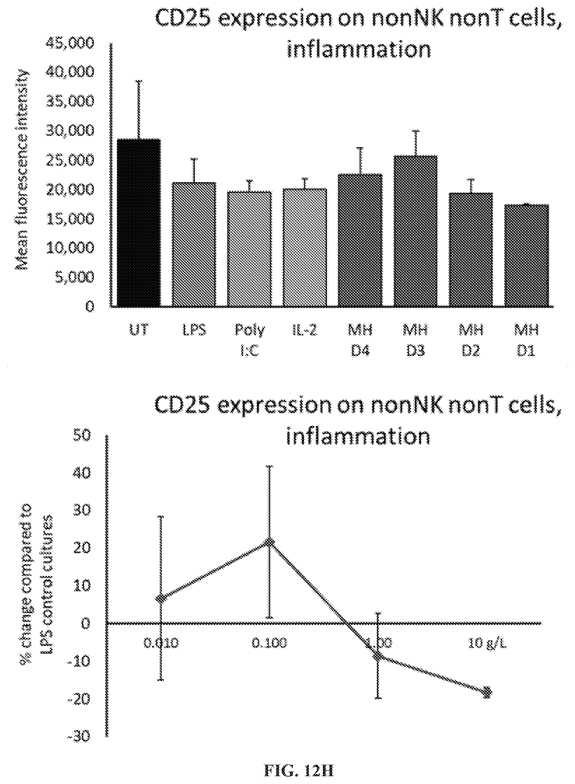

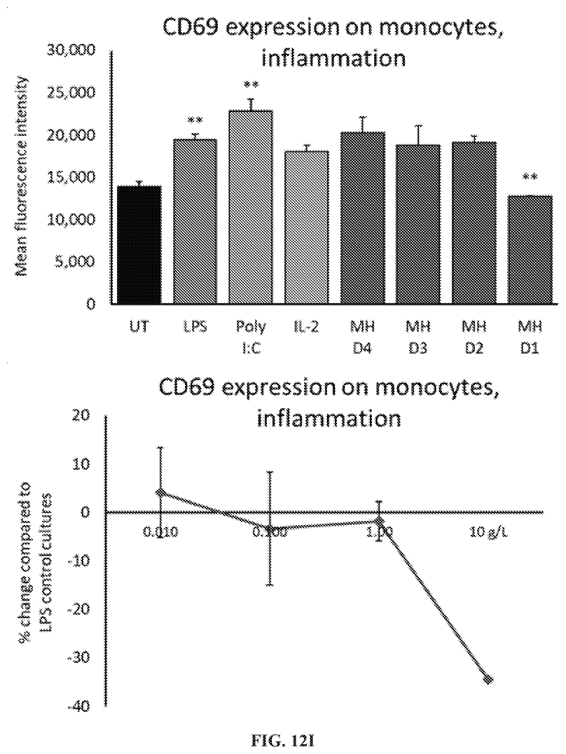

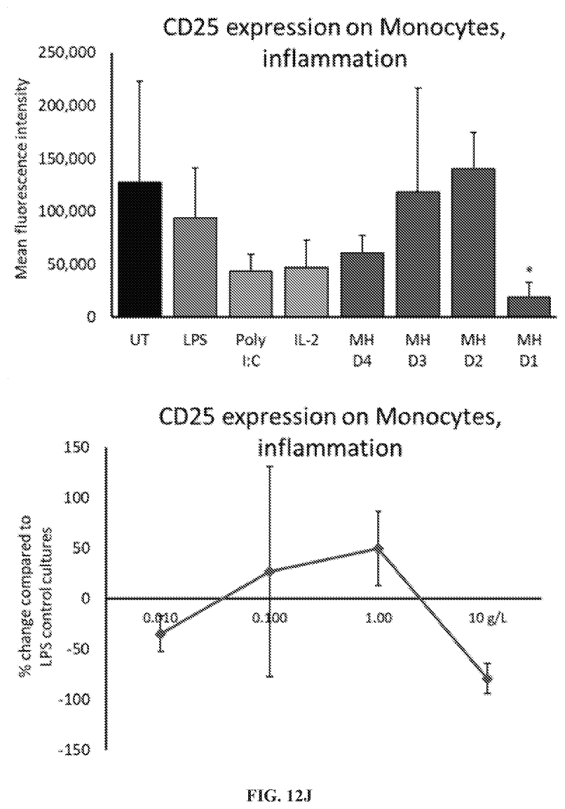

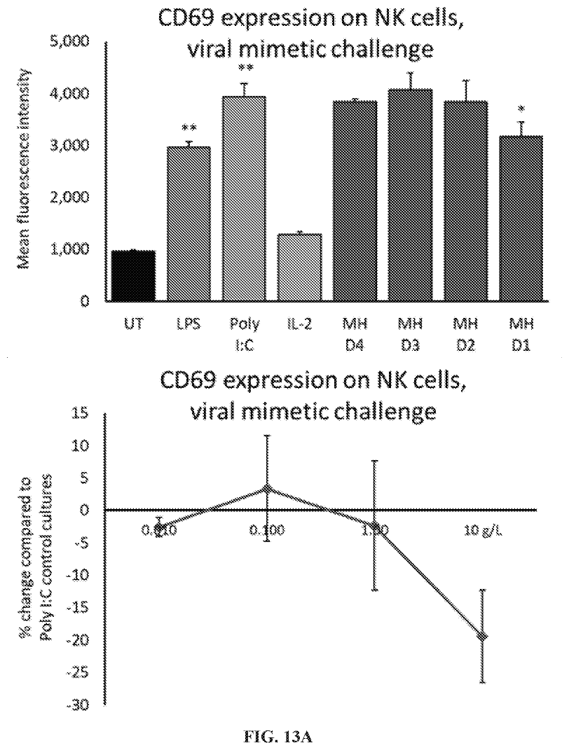

CD69 and CD25 expression was also measured under inflamed conditions. The extract led to a significant decrease in CD69 expression and CD25 expression on NK cells (FIGS. 12A-12B) as well as on NKT cells (FIGS. 12C-12D), non-NK, non-T cells (FIGS. 12G-12H), and monocytes (FIGS. 12I-12J). There was an increase in CD69 and CD25 expression on T cells (FIGS. 12E-12F).

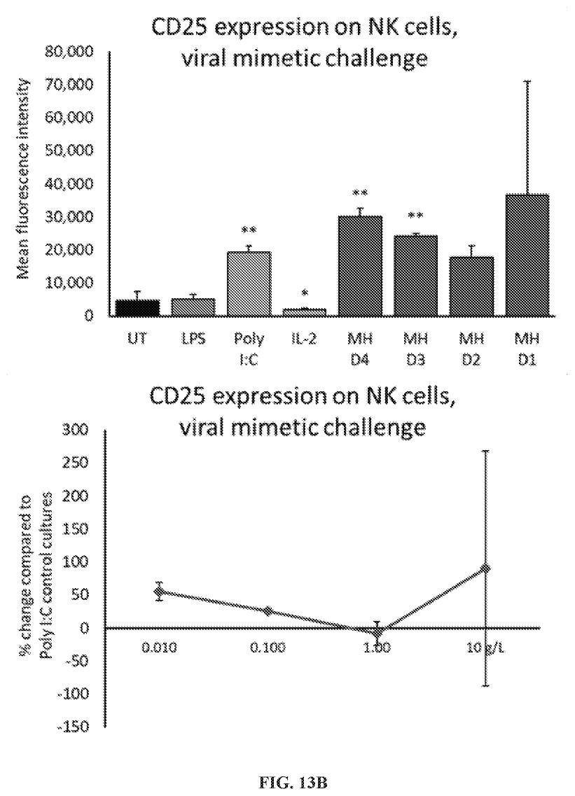

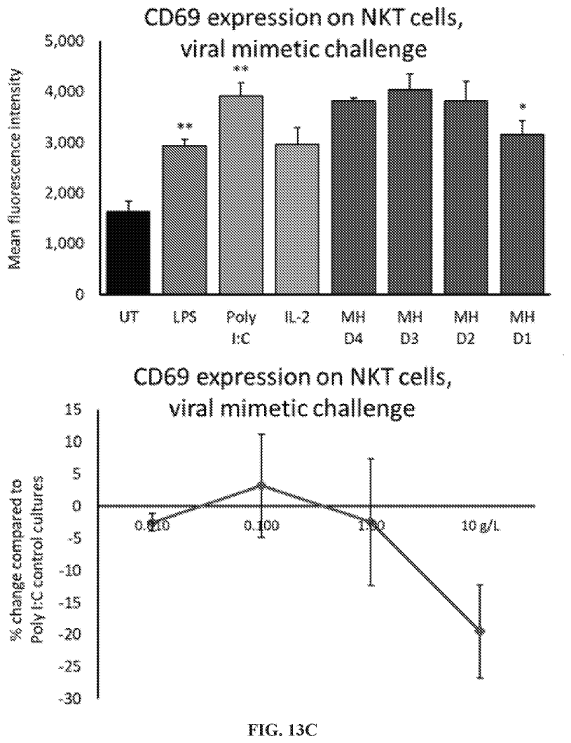

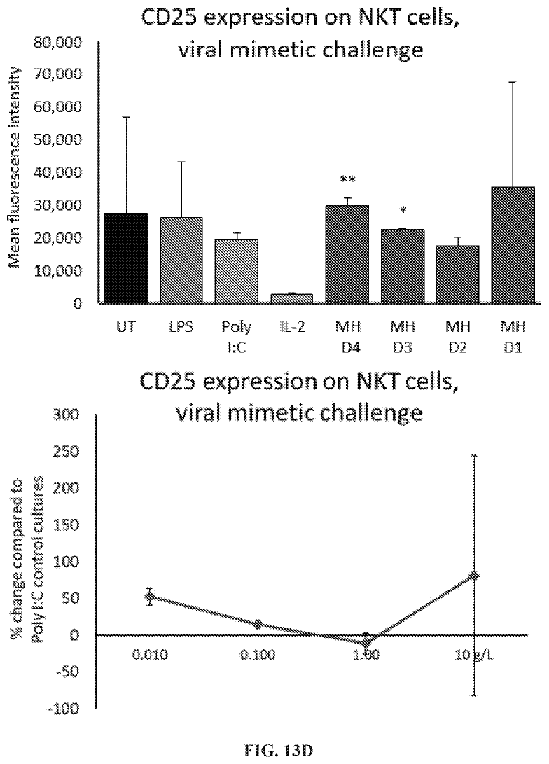

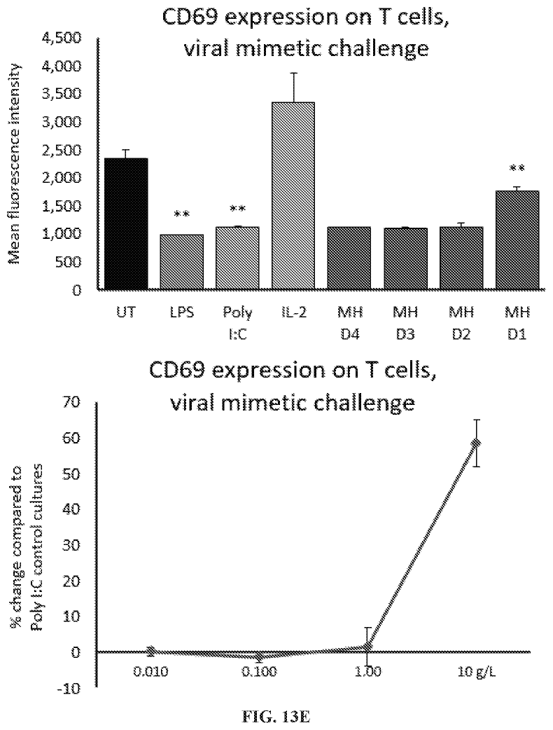

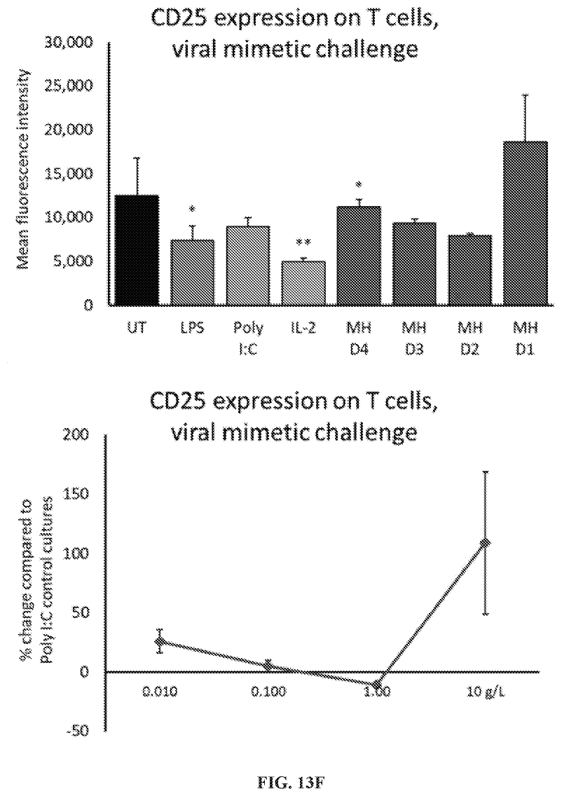

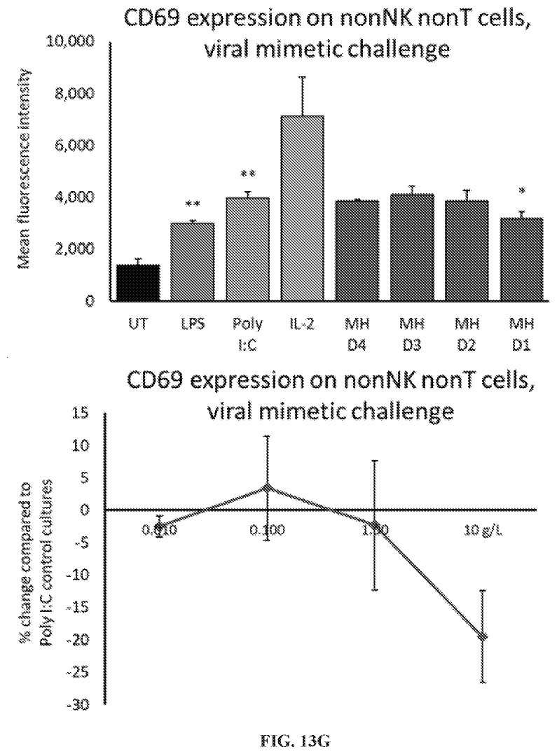

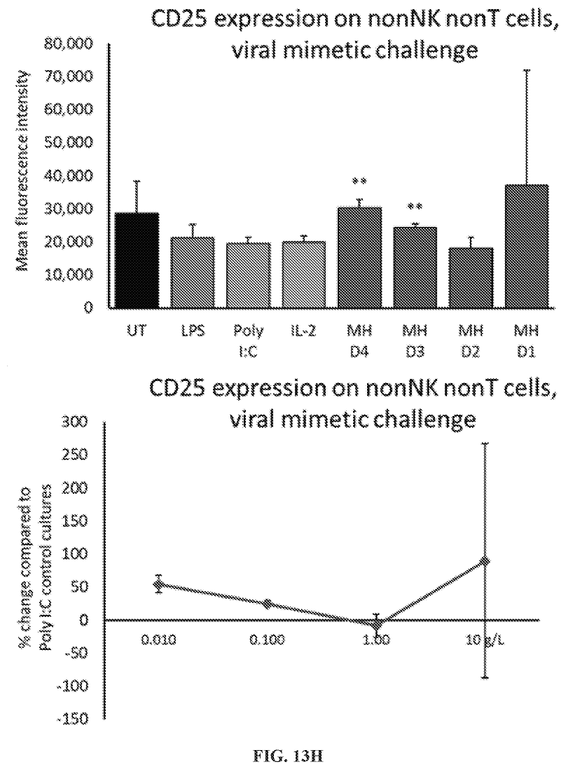

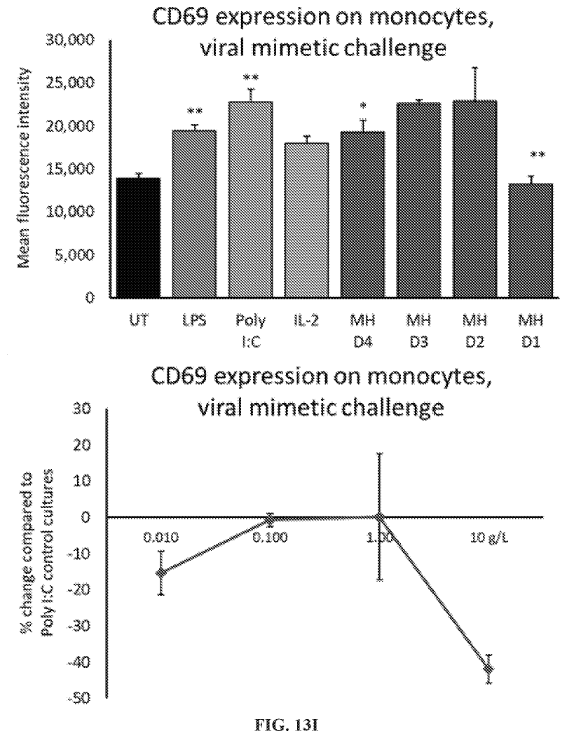

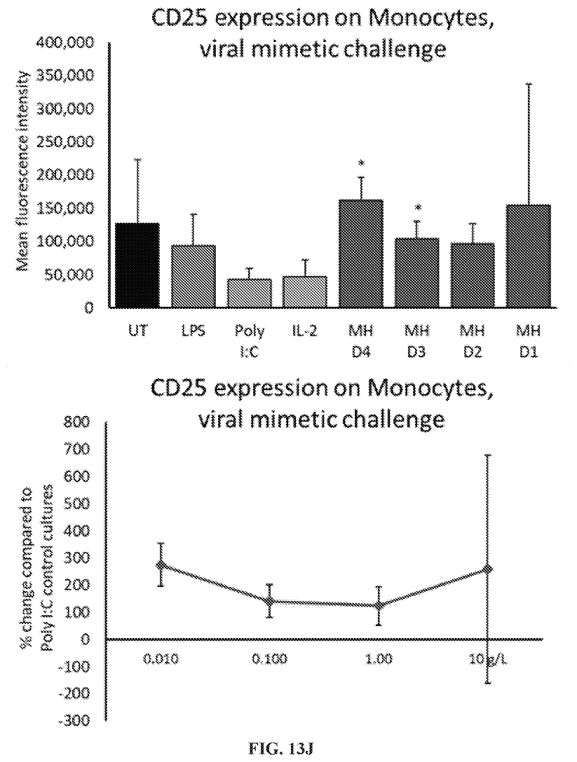

Finally, CD69 and CD25 expression was measured on the immune cells in the context of a viral mimetic insult. A decrease in CD69 expression and an increase in CD25 expression was observed on NK cells (FIGS. 13A-13B), NKT cells (FIGS. 13C-13D), non-NK, non-T cells (FIGS. 13G-13H), and monocytes (FIGS. 13I-13J). T cells showed an increase in CD69 and CD25 expression (FIGS. 13E-13F).

Thus, it was shown that the extract provides cellular oxidant protection as it was observed that the antioxidants are capable of penetrating into and protecting cells from oxidative stress. It was also shown that the extract had immune modulating effects in the context of inflammatory insult and viral mimetics.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Allen et al., “Remington: The Science and Practice of Pharmacy 22^{nd} ed.,” Pharmaceutical Press, Sep. 15, 2012.
Hornyak et al., “Introduction to Nanoscience and Nanotechnology,” CRC Press, 2008.
Singleton et al., “Dictionary of Microbiology and Molecular Biology 3^{rd} ed.,” revised ed., J. Wiley & Sons. 2006.
Smith, “March's Advanced Organic Chemistry Reactions, Mechanisms and Structure 7^{th} ed.,” J. Wiley & Sons Wiley-Blackwell, Nov. 28, 2012.
Green et al., “Molecular Cloning: A Laboratory Manual 4th ed.,” Cold Spring Harbor Laboratory Press, Cold Spring Harbor, N.Y., 2012.
Greenfield, “Antibodies A Laboratory Manual 2^{nd} ed.,” Cold Spring Harbor Press, Cold Spring Harbor N.Y., 2013.
Kohler et al, “Derivation of specific antibody-producing tissue culture and tumor lines by cell fusion,” Eur. J. Immunol., 6(7):511-9, July, 1976.
U.S. patent application Ser. No. 13/859,633
U.S. Pat. No. 5,585,089
Riechmann et al., “Reshaping human antibodies for therapy,” Nature, 332(6162):323-7, Mar. 24, 1988.
Yang and Kallio, “Fatty acid composition of lipids in sea buckthorn (Hippophae rhamnoides L.) berries of different origins,” J. Agr. Food Chem. 49:1939-1947, 2001.
Bal et al., “Sea buckthorn berries: A potential source of valuable nutrients for nutraceuticals and cosmeceuticals,” Food Res. Int., 44(7):1718-1727, 2011.
Tulsawani et al., “Efficacy of aqueous extract of Hippophae rhamnoides and its bio-active flavonoids against hypoxia-induced cell death,” Indian J. Pharmacol., 45(3):258-63, 2013.
Dhyani et al., “Basic nutritional attributes of Hippophae rhamnoides (sea buckthorn) populations from Uttarakhand Himalaya, India,” Curr. Sci. India. 92: 1148-1152, 2007.
Khan et al., “A comprehensive review of a magic plant Hippophae rhamnoides,” Pharmacogn. J. 16: 58-61, 2010.

What is claimed is:

1. A pharmaceutical composition comprising: one or more of the following components selected from the group consisting of: a Hippophae rhamnoides extract, a Ribes spp. extract; an Aphanizomenon flos aquae (AFA) extract; an Aloe extract; a Fucus vesiculosus extract; a Panar notoginseng extract; a beta-glucan and a pharmaceutically acceptable carrier.

2. The pharmaceutical composition of claim 1, comprising a Hippophae rhamnoides extract and at least one additional component selected from: a Ribes spp. extract; an Aphanizomenon flos aquae (AFA) extract; an Aloe extract; a Fucus vesiculosus extract; a Panax notoginseng extract; and a beta-glucan.

3. The pharmaceutical composition of claim 1, comprising a Hippophae rhamnoides extract and at least two additional components selected from: a Ribes spp. extract; an Aphanizomenon flos aquae (AFA) extract; a Aloe extract; a Fucus vesiculosus extract; a Panax notoginseng extract; and a beta-glucan.

4. The pharmaceutical composition of claim 1, comprising a Hippophae rhamnoides extract and at least three, four or five additional components selected from: a Ribes spp. extract; an Aphanizomenon flos aquae (AFA) extract; an Aloe extract; a Fucus vesiculosus extract; a Panax notoginseng extract; and a beta-glucan.

5. The pharmaceutical composition of claim 1, comprising a Hippophae rhamnoides extract and at least one, two, three, four or five additional components selected from: an Aphanizomenon flos aquae (AFA) extract; a Aloe extract; a Fucus vesiculosus extract; a Panax notoginseng extract; and a beta-glucan.

6. The pharmaceutical composition of anyone of claims 1-5, wherein the pharmaceutical composition comprises a Hippophae rhamnoides fruit or a Hippophae rhamnoides leaf extract.

7. The pharmaceutical composition of claim 6, wherein the pharmaceutical composition comprises a Hippophae rhamnoides leaf extract.

8. The pharmaceutical composition of claim 5, wherein the pharmaceutical composition comprises a Hippophae rhamnoides fruit extract.

9. The pharmaceutical composition of either claim 5, wherein the Hippophae rhamnoides fruit extract or the Hippophae rhamnoides leaf extract is an extract enriched for polyphenol and/or proanthocyanidin compounds.

10. The pharmaceutical composition of claim 9, wherein the composition is a single unit dosage and comprises 150-1000 mg of Hippophae rhamnoides extract.

11. The pharmaceutical composition of anyone of claims 1-9, comprising an Aphanizomenon flos aquae (AFA) extract.

12. The pharmaceutical composition of claim 11, wherein the composition is a single unit dosage and comprises 250-1000 mg of Aphanizomenon flos aquae (AFA) extract.

13. The pharmaceutical composition of anyone of claims 1-9, comprising a Fucus vesiculosus extract.

14. The pharmaceutical composition of claim 13, wherein the composition is a single unit dosage and comprises 150-1000 mg of a Fucus vesiculosus extract.

15. The pharmaceutical composition of claim 13, wherein the Fucus vesiculosus extract comprises at least 20%, 25%, 30%, 35%, or 40% phlorotannins.

16. The pharmaceutical composition of anyone of claims 1-9, comprising a Panax notoginseng extract.

17. The pharmaceutical composition of claim 16, wherein the composition is a single unit dosage and comprises 100-500 mg of a Panax notoginseng extract.

18. The pharmaceutical composition of claim 16, wherein the Panax notoginseng extract comprises at least 20%, 25%, 30%, 35%, or 40% saponins.

19. The pharmaceutical composition of anyone of claims 1-9, comprising a beta-glucan.

20. The pharmaceutical composition of claim 16, wherein the composition is a single unit dosage and comprises 50-500 mg of a beta-glucan.

21. The pharmaceutical composition of anyone of claims 1-9, comprising colostrum.

22. The pharmaceutical composition of claim 21, wherein the composition is a single unit dosage and comprises 5-100 mg of colostrum.

23. The pharmaceutical composition of anyone of claims 1-22, comprising a Hippophae rhamnoides extract; an Aphanizomenon flos aquae (AFA) extract; an Aloe extract; a Fucus vesiculosus extract; a Panax notoginseng extract; and a beta-glucan.

24. The pharmaceutical composition of anyone of claims 1-22, comprising a Hippophae rhamnoides extract; a Ribes spp. extract; an Aphanizomenon flos aquae (AFA) extract; an Aloe extract; a Fucus vesiculosus extract; a Panax notoginseng extract; and a beta-glucan.

25. The pharmaceutical composition of anyone of claims 1-21, comprising a Ribes spp. Extract.

26. The pharmaceutical composition of claim 1, comprising a Ribes nigrum and/or Ribes rubrum fruit extract.

27. The pharmaceutical composition of claim 26, wherein the Ribes nigrum and/or Ribes rubrum fruit extract is enriched for polyphenol compounds.

28. The pharmaceutical composition of claim 1, wherein the pharmaceutical composition comprises a polyphenol extract of Ribes nigrum and/or Ribes rubrum.

29. The pharmaceutical composition of claim 1, wherein the composition further comprises an Aloe extract, an Fucus vesiculosus extract, and/or an Aphanizomenon flos aquae extract.

30. The pharmaceutical composition of claim 29, wherein the composition comprises an Fucus vesiculosus extract comprising fucoidan and at least 5%, 10%, 20% or 30% phlorotannins content.

31. The pharmaceutical composition of claim 29, wherein the composition comprises an Aloe extract that has been enriched for poly acetyl mannan content.

32. The pharmaceutical composition of claim 31, wherein the Aloe extract comprises at least 10%, 15%, or 20% poly acetyl mannans.

33. The pharmaceutical composition of claim 29, wherein the composition is a single unit dosage and comprises 100-400 mg of Aloe extract.

34. The pharmaceutical composition of anyone of claims 1-33, comprising a Terminalia chebula extract.

35. The pharmaceutical composition of claim 34, comprising a Terminalia chebula fruit extract or a Terminalia chebula leaf extract.

36. The pharmaceutical composition of claim 35, comprising a Terminalia chebula leaf extract.

37. The pharmaceutical composition of claim 35, comprising a Terminalia chebula fruit extract.

38. The pharmaceutical composition of claim 35, wherein the Terminalia chebula fruit or Terminalia chebula leaf extract is enriched for tannin compounds.

39. The pharmaceutical composition of claim 34, wherein the pharmaceutical composition comprises a tannin extract of Terminalia chebula.

40. The pharmaceutical composition of claim 39, wherein the Terminalia chebula fruit extract or the Terminalia chebula leaf extract comprises at least 10%, 20%, 30% or 40% tannins.

41. The pharmaceutical composition of claim 1, wherein the composition is sterile.

42. The pharmaceutical composition of claim 1, wherein the composition is formulated for oral administration.

43. The pharmaceutical composition of claim 1, wherein the composition is comprised in a capsule.

44. The pharmaceutical composition of claim 32, where the capsule comprises an enteric coating.

45. The pharmaceutical composition of claim 43, wherein the capsule comprises about 50 to 100, 200, 250, 500 or 750 mg of the composition.

46. The pharmaceutical composition of claim 43, comprising between about 50 and 750 mg of Hippophae rhamnoides extract.

47. The pharmaceutical composition of claim 43, comprising between about 50 and 250 mg of Panax notoginseng extract.

48. A method of increasing stem cell mobilization in a subject, comprising administering to the subject a sufficient amount of a composition comprising a Hippophae rhamnoides extract to provide stem cell mobilization to the blood.

49. The method of claim 48, comprising administering a composition in accordance with any one of claims 1-44.

50. The method of claim 52, wherein the composition comprises a Hippophae rhamnoides fruit extract or a Hippophae rhamnoides leaf extract.

51. The method of claim 53, wherein the composition comprises a Hippophae rhamnoides leaf extract.

52. The method of claim 53, wherein the composition comprises a Hippophae rhamnoides fruit extract.

53. The method of claim 50, wherein the mobilization agent comprises a Hippophae rhamnoides fruit extract or a Hippophae rhamnoides leaf extract that is enriched for polyphenols and/or proanthocyanidins.

54. The method of claim 48, wherein the mobilization agent comprises a Terminalia chebula fruit extract or a Terminalia chebula leaf extract.

55. The method of claim 54, wherein the mobilization agent comprises a Terminalia chebula fruit extract or a Terminalia chebula leaf extract comprising at least 10%, 20%, 30% or 40% tannins.

56. The method of claim 48, wherein the composition further comprises and Aloe extract, an Echinacea purpurea extract or an Aphanizomenon jlos aquae extract.

57. The method of claim 48, wherein the mobilization agent comprises an Echinacea purpurea extract comprising cichoric acid and at least 1%, 2%, 3% or 4% polyphenol content.

58. The method of claim 48, wherein the stem cell comprises a bone marrow-derived stem cell (BMSC).

59. The method of claim 48, wherein the stem cell comprises a hematopoietic stem cell (HSC).

60. The method of claim 48, wherein administering the composition comprises oral administration.

61. The method of claim 60, wherein oral administration is more than once a day.

62. The method of claim 60, wherein oral administration is daily.

63. The method of claim 60, wherein the composition is comprised in a capsule.

64. The method of claim 63, where the capsule comprises an enteric coating.

65. The method of claim 63, wherein the capsule comprises about 50 to 100, 200, 250, or 500 mg of the composition.

66. The method of claim 63, wherein the capsule comprises about 250 to 500, 750, or 1000 mg of the composition.

67. The method of claim 66, comprising administering about 50 to 750 mg of the Hippophae rhamnoides fruit extract or the Hippophae rhamnoides leaf extract.

68. The method of claim 66, comprising administering about 50 to 750 mg of the Terminalia chebula fruit extract or the Terminalia chebula leaf extract.

69. The method of claim 48, comprising administering a composition in accordance with any one of claims 1-44.

70. A pharmaceutical composition comprising: one or more of the following components selected from the group consisting of: a Hippophae rhamnoides leaf extract and Terminalia chebula leaf extract; and a pharmaceutically acceptable carrier.

71. The pharmaceutical composition of claim 70, comprising a Hippophae rhamnoides leaf extract and a Terminalia chebula leaf extract.

72. The pharmaceutical composition of claim 70, wherein the pharmaceutical composition comprises a Hippophae rhamnoides leaf extract.

73. The pharmaceutical composition of either claim 72, wherein the Hippophae rhamnoides leaf extract is an extract enriched for polyphenol and/or proanthocyanidin compounds.

74. The pharmaceutical composition of claim 70, comprising a Terminalia chebula leaf extract.

75. The pharmaceutical composition of claim 74, wherein the Terminalia chebula fruit or Terminalia chebula leaf extract is enriched for tannin compounds.

76. The pharmaceutical composition of claim 70, wherein the pharmaceutical composition comprises a tannin extract of Terminalia chebula.

77. The pharmaceutical composition of claim 76, wherein the Terminalia chebula leaf extract comprises at least 10%, 20%, 30%, or 40% tannins.

78. The pharmaceutical composition of claim 70, wherein the composition further comprises an Aloe extract, an Echinacea purpurea extract, and/or an Aphanizomenon jlos aquae extract.

79. The pharmaceutical composition of claim 78, wherein the composition comprises an Echinacea purpurea extract comprising cichoric acid and at least 1%, 2%, 3% or 4% polyphenol content.

80. The pharmaceutical composition of claim 78, wherein the composition comprises an Aloe extract that has been enriched for poly acetyl mannan content.

81. The pharmaceutical composition of claim 80, wherein the Aloe extract comprises at least 10%, 15%, or 20% poly acetyl mannans.

82. The pharmaceutical composition of claim 70, wherein the composition is sterile.

83. The pharmaceutical composition of claim 70, wherein the composition is formulated for oral administration.

84. The pharmaceutical composition of claim 70, wherein the composition is comprised in a capsule.

85. The pharmaceutical composition of claim 84, where the capsule comprises an enteric coating.

86. The pharmaceutical composition of claim 84, wherein the capsule comprises about 50 to 100, 200, 250, or 500 mg of the composition.

87. The pharmaceutical composition of claim 84, comprising between about 50 and 750 mg of Hippophae rhamnoides leaf extract.

88. The pharmaceutical composition of claim 84, comprising between about 50 and 750 mg of Terminalia chebula leaf extract.

89. A method of increasing stem cell mobilization in a subject, comprising administering to the subject a sufficient amount of a composition comprising a Hippophae rhamnoides leaf extract and/or a Terminalia chebula leaf extract to provide stem cell mobilization to the blood.

90. The method of claim 89, wherein the composition comprises a Hippophae rhamnoides leaf extract.

91. The method of claim 89, wherein the mobilization agent comprises a Hippophae rhamnoides leaf extract that is enriched for polyphenols and/or proanthocyanidins.

92. The method of claim 89, wherein the mobilization agent comprises a Terminalia chebula leaf extract.

93. The method of claim 92, wherein the mobilization agent comprises a Terminalia chebula leaf extract comprising at least 10%, 20%, 30%, or 40% tannins.

94. The method of claim 89, wherein the composition further comprises and Aloe extract, an Echinacea purpurea extract or an Aphanizomenon jlos aquae extract.

95. The method of claim 89, wherein the mobilization agent comprises an Echinacea purpurea extract comprising cichoric acid and at least 1%, 2%, 3%, or 4% polyphenol content.

96. The method of claim 89, wherein the stem cell comprises a bone marrow-derived stem cell (BMSC).

97. The method of claim 89, wherein the stem cell comprises a hematopoietic stem cell (HSC).

98. The method of claim 89, wherein administering the composition comprises oral administration.

99. The method of claim 98, wherein oral administration is more than once a day.

100. The method of claim 98, wherein oral administration is daily.

101. The method of claim 98, wherein the composition is comprised in a capsule.

102. The method of claim 101, where the capsule comprises an enteric coating.

103. The method of claim 101, wherein the capsule comprises about 50 to 100, 200, 250, or 500 mg of the composition.

104. The method of claim 101, wherein the capsule comprises about 250 to 500, 750, or 1000 mg of the composition.

105. The method of claim 104, comprising administering about 50 to 750 mg of the Hippophae rhamnoides leaf extract.

106. The method of claim 104, comprising administering about 50 to 750 mg of the Terminalia chebula leaf extract.

107. The method of claim 89, comprising administering a composition in accordance with any one of claims 70-88.

108. A method of decreasing oxidative stress and/or inflammation in a subject, comprising administering to the subject a sufficient amount of a composition comprising a Ribes spp. extract and/or a Hippophae rhamnoides extract to provide antioxidant and/or anti-inflammatory effects to the cells of said subject.

109. The method of claim 108, comprising administering a composition in accordance with any one of claims 1-44.

110. The method of claim 108, comprising administering a Ribes nigrum and/or Ribes rubrum fruit extract.

111. The method of claim 110, wherein the Ribes nigrum and/or Ribes rubrum fruit extract is enriched for polyphenol compounds.

112. The method of claim 109, wherein the composition comprises a Hippophae rhamnoides fruit extract or a Hippophae rhamnoides leaf extract.

113. The method of claim 109, wherein the composition comprises a Hippophae rhamnoides leaf extract.

114. The method of claim 109, wherein the composition comprises a Hippophae rhamnoides fruit extract.

115. The method of claim 108, wherein the composition comprises a Terminalia chebula fruit extract or a Terminalia chebula leaf extract.

116. The method of claim 115, wherein the composition comprises a Terminalia chebula fruit extract or a Terminalia chebula leaf extract comprising at least 10%, 20%, 30% or 40% tannins.

117. The method of claim 108, wherein the composition further comprises and Aloe extract, an Echinacea purpurea extract or an Aphanizomenon jlos aquae extract.

118. The method of claim 1088, wherein the composition comprises an Echinacea purpurea extract comprising cichoric acid and at least 1%, 2%, 3% or 4% polyphenol content.

119. The method of claim 108, wherein the composition results in decreased oxidative stress in red blood cells.

120. The method of claim 119, wherein the composition penetrates the cell membrane.

121. The method of claim 108, wherein the composition results in decreased reactive oxygen species (ROS) formation.

122. The method of claim 108, wherein administering the composition comprises oral administration.

123. The method of claim 122, wherein oral administration is more than once a day.

124. The method of claim 122, wherein oral administration is daily.

125. The method of claim 122, wherein the composition is comprised in a capsule.

126. The method of claim 125, wherein the capsule comprises an enteric coating.

127. The method of claim 125, wherein the capsule comprises about 50 to 100, 200, 250, or 500 mg of the composition.

128. The method of claim 125, wherein the capsule comprises about 250 to 500, 750, or 1000 mg of the composition.

129. The method of claim 128, comprising administering about 50 to 750 mg of the Hippophae rhamnoides fruit extract or the Hippophae rhamnoides leaf extract.

130. The method of claim 128, comprising administering about 50 to 750 mg of the Terminalia chebula fruit extract or the Terminalia chebula leaf extract.

131. A method of modulating immune response in a subject, comprising administering to the subject a sufficient amount of a composition comprising a Ribes spp. extract and/or a Hippophae rhamnoides extract to provide immune modulatory effects to the cells of said subject.

132. The method of claim 131, comprising administering a composition in accordance with any one of claims 1-44.

133. The method of claim 131, comprising administering a Ribes nigrum and or Ribes rubrum fruit extract.

134. The method of claim 133, wherein the Ribes nigrum and or Ribes rubrum fruit extract is enriched for polyphenol compounds.

135. The method of claim 132, wherein the composition comprises a Hippophae rhamnoides fruit extract or a Hippophae rhamnoides leaf extract.

136. The method of claim 132, wherein the composition comprises a Hippophae rhamnoides leaf extract.

137. The method of claim 132, wherein the composition comprises a Hippophae rhamnoides fruit extract.

138. The method of claim 131, wherein the composition comprises a Terminalia chebula fruit extract or a Terminalia chebula leaf extract.

139. The method of claim 138, wherein the composition comprises a Terminalia chebula fruit extract or a Terminalia chebula leaf extract comprising at least 10%, 20%, 30% or 40% tannins.

140. The method of claim 131, wherein the composition further comprises and Aloe extract, an Echinacea purpurea extract or an Aphanizomenon jlos aquae extract.

141. The method of claim 131, wherein the composition comprises an Echinacea purpurea extract comprising cichoric acid and at least 1%, 2%, 3% or 4% polyphenol content.

142. The method of claim 131, wherein the composition results in activation of lymphocytes towards an effector state.

143. The method of claim 142, wherein the composition results in an increase of CD69 expression on natural killer (NK) cells, natural killer T (NKT) cells, T cells, B lymphocytes, and/or dendritic cells as compared to CD69 expression before administration of the composition.

144. The method of claim 131, wherein the composition modulates immune response towards virally-infected cells or cancer cells.

145. The method of claim 144, wherein the composition results in increased CD69 expression on T cells.

146. The method of claim 131, wherein administering the composition comprises oral administration.

147. The method of claim 146, wherein oral administration is more than once a day.

148. The method of claim 146, wherein oral administration is daily.

149. The method of claim 146, wherein the composition is comprised in a capsule.

150. The method of claim 149, wherein the capsule comprises an enteric coating.

151. The method of claim 149, wherein the capsule comprises about 50 to 100, 200, 250, or 500 mg of the composition.

152. The method of claim 149, wherein the capsule comprises about 250 to 500, 750, or 1000 mg of the composition.

153. The method of claim 152, comprising administering about 50 to 750 mg of the Hippophae rhamnoides fruit extract or the Hippophae rhamnoides leaf extract.

154. The method of claim 152, comprising administering about 50 to 750 mg of the Terminalia chebula fruit extract or the Terminalia chebula leaf extract.
